(19) United States
(12) Patent Application Publication
(10) Pub. No.: US 2004/0223949 A1
(43) Pub. Date: Nov. 11, 2004

(54) Vaccines using high-dose cytokines

(75) Inventors: Igor Astsaturov, Baltimore, MD (US); Teresa Petrella, North York (CA); Mark DeBenedette, Toronto (CA); Neil Berinstein, Toronto (CA); David E. Spaner, Toronto (CA)

Correspondence Address: Patrick J. Halloran, Aventis Pasteur, Knerr Building, Discovery Drive, Swiftwater, PA, 18370, US

(73) Assignee: Sunnybrook and Women's College Health Sciences Center Aventis Pasteur, Ltd.

(21) Appl. No.: 10/690,199

(22) Filed: Oct. 21, 2003

Related U.S. Application Data
(60) Provisional application No. 60/420,425, filed on Oct. 22, 2002.

(51) Int. Cl. A61K 38/21; A61K 48/00; A61K 39/00

(52) U.S. Cl. 424/85.7; 424/93.2; 424/185.1

(57) ABSTRACT

The present invention relates to the field of cancer immunotherapy. In particular, vaccines are administered in conjunction with high doses of cytokines to enhance an anti-tumor immune response.

### RELATED APPLICATIONS

This application claims priority to U.S. Ser. No. 60/420,425 filed Oct. 22, 2002.

### FIELD OF THE INVENTION

The present invention relates to the field of cancer immunotherapy. In particular, vaccines are administered in conjunction with high doses of cytokines to enhance an anti-tumor immune response.

### BACKGROUND OF THE INVENTION

The present invention relates to a method for treating cancer by vaccinating a patient against cancer and following vaccination with administration of a cytokine in high doses. In particular, the invention relates to immunization against a tumor antigen followed by treatment with a T cell activating cytokine such as IFN-α.

Cancers, such as melanoma, that are incurable with conventional chemotherapy may be susceptible to treatment with vaccines that enhance the activity of tumor-reactive T cells. In the last few years, a number of tumor antigens have been identified and used to make specific cancer vaccines. For melanoma, these antigens include members of the MAGE family, tyrosinase, gp100, melanA/Mart-1, and Trp-2.

Despite the identification of these defined tumor targets, therapeutic results with cancer vaccines have been largely disappointing. While it has been relatively simple to transiently activate tumor-reactive T cells with vaccines, these responses are often not maintained for sufficient time to provide significant therapeutic benefits. Reasons for this transient activation include: i. tumor-reactive T cells are often only weakly reactive to tumor antigens that are self-antigens; ii. T cells exposed to these antigens during tumor progression may have become anergic; or, iii. immunoregulatory controls that prevent sustained auto-immune responses may also inhibit anti-tumor responses.

There is a need in the art for reagents and methodologies useful in stimulating an strong and consistent anti-cancer immune response treat cancer. The present invention provides such reagents and methodologies that overcome many of the difficulties encountered by others in attempting to treat cancer.

### SUMMARY OF THE INVENTION

The present invention provides a method for treating cancer by administering high doses of at least one cytokine following immunization against at least one tumor antigen. In one embodiment, the cytokine IFN-α is combined with a gp100 DNA-based vaccine to increase the anti-gp100 immune response in a cancer patient.

### BRIEF DESCRIPTION OF THE DRAWINGS

FIG. 1: High-dose IFN-α recalls tumor-reactive T cells previously activated by vaccines. PBMC from patients M166 AND M335 were stimulated with the mixture of HLA-A*0201 binding gp100 peptides (gp100:209-2M and gp100:280-9V) for 8 days as described in the materials and methods. The cells were then stained with the respective phycoerythrin-labeled tetramers and CD8-FITC antibodies and analyzed by flow cytometry. The percentage of CD8⁺tetramer⁺ cells, representing gp100-reactive T cells, was then determined and is indicated in the box in each dot-plot. Before starting the schedule of vaccinations, very few gp100-reactive T cells were found in both patients (baseline). Both patients responded to vaccination and the peak response is shown in the dot-plot marked “on vaccine”. Before commencing HDI, gp100-reactive T cell numbers had essentially returned to baseline (follow-up). Two weeks (for M166) and 3 weeks (for M335) after starting HDI, the number of gp100-reactive T cells again increased. All cultures were carried out at the same time, using blood that had been obtained at the indicated times and then cryopreserved.

FIG. 2: Clinical response to HDI of patient M166. Magnetic resonance imaging (MRI) studies of a gluteal mass (arrows), presumed on clinical and radiologic grounds to be metastatic melanoma, before vaccination (a), 3 months after completing the vaccination protocol (b), and one month after completing HDI (c). The mass was somewhat smaller after completing the vaccine protocol but essentially disappeared after HDI.

FIG. 3: Increased numbers of gp100-reactive T cells after HDI measured by IFN-γ ELISPOT assays in M166. Following the final vaccination, PBMC were collected monthly for 3 months (a) and then before beginning HDI, weekly while on HDI (indicated by the double arrow), and then monthly for 3 months (b,c). The samples were then cryopreserved so that they could all be analyzed at the same time. The cells were thawed and stimulated with a mix of the gp100 peptides (gp100:209-2M and gp100:280-9V) or FLU MP peptides (c), as a control to ensure that the culture conditions were adequate to reveal memory T cells if they were present. After 8 days of culture, ELISPOT assays were performed as described in the materials and methods. Cells were reactivated on ELISPOT plates with the gp100 peptides (solid black bar) or FLU peptides (gray bar). The average and standard deviation of the number of spots from 3 replicate wells are reported.

FIG. 4: Clinical response to HDI of patient M335. Computerized axial tomography (CAT) scans of left axillary adenopathy (a, b, c) (arrows) and a lung nodule (d, e, f) (arrows), presumed to represent metastatic melanoma on clinical and radiologic grounds, before vaccination (a, d), one month after completing the vaccination protocol (b, e) and one month after completing HDI (c, f). Both areas of involvement progressed through the vaccine schedule but regressed considerably after HDI.

FIG. 5: Increased numbers of gp100-reactive T cells after HDI measured by IFN-γ ELISPOT assays in M335. PBMC were collected monthly during active vaccination (months-3 and -2, indicated by the double-headed arrow labeled “vaccine”), an observation period (months-1 and 0), weekly for 4 weeks on HDI (indicated by the double-headed arrow labeled “IFNα2b”), and then for one month following completion of HDI (month 2). Cryopreserved cells were thawed and stimulated with the two gp100peptides. After 8 days of culture, the cells were harvested and reactivated on IFN-γ antibody coated ELISPOT plates with either the gp100 peptides (black bars) or the control FLU peptides (gray bars), as described in the materials and methods. The average and standard deviation of the number of spots from 3 replicate wells is reported.

FIG. 6: Enhanced killing activity of gp100-reactive T cells during HDI. Cryopreserved PBMC after vaccination, during HDI, and one month after HDI from M166 (a) and after vaccination and one month post HDI from M335 were stimulated with the gp100-209-2M and gp100:280-9V peptides for 8 days. The cells were harvested and cultured with 2000 chromium labeled T2 cells that had been coated with the gp100 peptides or with a control HIV peptide in the effector:target (E:T) ratios indicated on the X-axis. Chromium release was measured 4 hours later. The average and standard deviation of the percent lysis from 4 replicate wells is shown. Specific killing of gp100 peptide-coated tumor targets is only seen when HDI had been given to the patients. Direct addition of IFN-α to the cultures did not increase gp100-specific CTL activity (not shown). In (a), the graph on the right marked “Flu-post vaccine” shows the CTL activity on FLU peptide-coated T2 cells when the same PBMC from M166 had been activated by FLU peptides and indicates that the culture conditions could support specific CTL activity if it was present.

### DETAILED DESCRIPTION

The present invention provides reagents and methodologies useful for treating and/or preventing cancer. All references cited within this application are incorporated by reference.

In one embodiment, the present invention relates to the induction or enhancement of an immune response against one or more tumor antigens (“TA”) to prevent and/or treat cancer. In certain embodiments, one or more TAs may be combined. In preferred embodiments, the immune response results from expression of a TA in a host cell following administration of a nucleic acid vector encoding the tumor antigen or the tumor antigen itself in the form of a peptide or polypeptide, for example.

As used herein, an “antigen” is a molecule (such as a polypeptide) or a portion thereof that produces an immune response in a host to whom the antigen has been administered. The immune response may include the production of antibodies that bind to at least one epitope of the antigen and/or the generation of a cellular immune response against cells expressing an epitope of the antigen. The response may be an enhancement of a current immune response by, for example, causing increased antibody production, production of antibodies with increased affinity for the antigen, or an increased cellular response (i.e., increased T cells). An antigen that produces an immune response may alternatively be referred to as being immunogenic or as an immunogen. In describing the present invention, a TA may be referred to as an “immunogenic target”.

TA includes both tumor-associated antigens (TAAs) and tumor-specific antigens (TSAs), where a cancerous cell is the source of the antigen. A TAA is an antigen that is expressed on the surface of a tumor cell in higher amounts than is observed on normal cells or an antigen that is expressed on normal cells during fetal development. A TSA is an antigen that is unique to tumor cells and is not expressed on normal cells. TA further includes TAAs or TSAs, antigenic fragments thereof, and modified versions that retain their antigenicity.

TAs are typically classified into five categories according to their expression pattern, function, or genetic origin: cancer-testis (CT) antigens (i.e., MAGE, NY-ESO-1); melanocyte differentiation antigens (i.e., Melan A/MART-1, tyrosinase, gp100); mutational antigens (i.e., MUM-1, p53, CDK-4); overexpressed ‘self’ antigens (i.e., HER-2/neu, p53); and, viral antigens (i.e., HPV, EBV). For the purposes of practicing the present invention, a suitable TA is any TA that induces or enhances an anti-tumor immune response in a host to whom the TA has been administered. Suitable TAs include, for example, gp100 (Cox et al., Science, 264:716-719 (1994)), MART-1/Melan. A (Kawakami et al., J. Exp. Med., 180:347-352 (1994)), gp75 (TRP-1) (Wang et al., J. Exp. Med., 186:1131-1140 (1996)), tyrosinase (Wolfel et al., Eur. J Immunol., 24:759-764 (1994); WO 200175117; WO 200175016; WO 200175007), NY-ESO-1 (WO 98/14464; WO 99/18206), melanoma proteoglycan (Hellstrom et al., J. Immunol., 130:1467-1472 (1983)), MAGE family antigens (i.e., MAGE-1, 2,3,4,6,12, 51; Van der Bruggen et al., Science, 254:1643-1647 (1991); U.S. Pat. No. 6,235,525; CN 1319611), BAGE family antigens (Boel et al., Immunity, 2:167-175 (1995)), GAGE family antigens (i.e., GAGE-1,2; Van den Eynde et al., J. Exp. Med., 182:689-698 (1995); U.S. Pat. No., 6,013,765), RAGE family antigens (i.e., RAGE-1; Gaugler et at., Immunogenetics, 44:323-330 (1996); U.S. Pat. No. 5,939,526), N-acetylglucosaminyltransferase-V (Guilloux et at., J. Exp. Med., 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad. Sci. USA, 92:7976-7980 (1995)), cyclin dependent kinase-4 (CDK4) (Wolfel et al., Science, 269:1281-1284 (1995)), p21-ras (Fossum et at., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), p185 HER2/neu (erb-B1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome et al., J. Immunol., 151:1654-1662 (1993)); EBNA gene products of EBV (i.e., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun 2000 Sep 7;275(3):731-8), HIP-55, TGFβ-1 anti-apoptotic factor (Toomey, et al. Br J Biomed Sci 2001;58(3):177-83), tumor protein D52 (Bryne J. A., et al., Genomics, 35:523-532 (1996)), H1FT, NY-BR-1 (WO 01/47959), NY-BR-62, NY-BR-75, NY-BR-85, NY-BR-87, NY-BR-96 (Scanlan, M. Serologic and Bioinformatic Approaches to the Identification of Human Tumor Antigens, in Cancer Vaccines 2000, Cancer Research Institute, New York, N.Y.), including “wild-type” (i.e., normally encoded by the genome, naturally-occurring), modified, and mutated versions as well as other fragments and derivatives thereof. Any of these TAs may be utilized alone or in combination with one another in a co-immunization protocol.

In certain cases, it may be beneficial to co-immunize patients with both TA and other antigens, such as angiogenesis-associated antigens (“AA”). An AA is an immunogenic molecule (i.e., peptide, polypeptide) associated with cells involved in the induction and/or continued development of blood vessels. For example, an AA may be expressed on an endothelial cell (“EC”), which is a primary structural component of blood vessels. Where the cancer is cancer, it is preferred that that the AA be found within or near blood vessels that supply a tumor. Immunization of a patient against an AA preferably results in an anti-AA immune response whereby angiogenic processes that occur near or within tumors are prevented and/or inhibited.

Exemplary AAs include, for example, vascular endothelial growth factor (i.e., VEGF; Bernardini, et al. J. Urol., 2001, 166(4): 1275-9; Starnes, et al. J. Thorac. Cardiovasc. Surg., 2001, 122(3): 518-23; Dias, et al. Blood, 2002, 99: 2179-2184), the VEGF receptor (i.e., VEGF-R, flk-1/KDR; Starnes, et al. J. Thorac. Cardiovasc. Surg., 2001, 122(3): 518-23), EPH receptors (i.e., EPHA2; Gerety, et al. 1999, Cell, 4: 403-414), epidermal growth factor receptor (i.e., EGFR; Ciardeillo, et al. Clin. Cancer Res., 2001, 7(10): 2958-70), basic fibroblast growth factor (i.e., bFGF; Davidson, et al. Clin. Exp. Metastasis 2000,18(6): 501-7; Poon, et al. Am J. Surg., 2001, 182(3):298-304), platelet-derived cell growth factor (i.e., PDGF-B), platelet-derived endothelial cell growth factor (PD-ECGF; Hong, et al. J. Mol. Med., 2001, 8(2):141-8), transforming growth factors (i.e., TGF-α; Hong, et al. J. Mol. Med., 2001, ₈(₂):₁₄1-₈), endoglin (Balza, et al. Int. J. Cancer, 2001, 94: 579-585), Id proteins (Benezra, R. Trends Cardiovasc. Med., 2001, 11(6):237-41), proteases such as uPA, uPAR, and matrix metalloproteinases (MMP-2, MMP-9; Djonov, et al. J. Pathol., 2001, 195(2):147-55), nitric oxide synthase (Am. J. Ophthalmol., 2001, 132(4):551-6), aminopeptidase (Rouslhati, E. Nature Cancer, 2: 84-90, 2002), thrombospondins (i.e., TSP-1, TSP-2; Alvarez, et al. Gynecol. Oncol., 2001, 82(2):273-8; Seki, et al. Int. J. Oncol., 2001, 19(2):305-10), k-ras (Zhang, et al. Cancer Res., 2001, 61(16):6050-4), Wnt (Zhang, et al. Cancer Res., 2001, 61(16):6050-4), cyclin-dependent kinases (CDKs; Drug Resist. Updat. 2000, 3(2):83-88), microtubules (Timar, et al. 2001. Path. Oncol. Res., 7(2): 85-94) heat shock proteins (i.e., HSP90 (Timar, supra)), heparin-binding factors (i.e., heparinase; Gohji, et al. Int. J. Cancer, 2001, 95(5):295-301), synthases (i.e., ATP synthase, thymidilate synthase), collagen receptors, integrins (i.e., αυβ3, αυβ5, α1β1, α2⊕1, α5⊕1), the surface proteolglycan NG2, among others, including “wild-type” (i.e., normally encoded by the genome, naturally-occurring), modified, mutated versions as well as other fragments and derivatives thereof. Any of these targets may be suitable in practicing the present invention, either alone or in combination with one another or with other agents.

In certain embodiments, a nucleic acid molecule encoding an immunogenic target is utilized. The nucleic acid molecule may comprise or consist of a nucleotide sequence encoding one or more immunogenic targets, or fragments or derivatives thereof, such as that contained in a DNA insert in an ATCC Deposit. The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N6-methyladenosine, aziridinyl-cytosine, pseudoisocytosine, 5-(carboxyhydroxylmethyl)uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1 -methylguanine, 1 -methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine, among others.

An isolated nucleic acid molecule is one that: (1) is separated from at least about 50 percent of proteins, lipids, carbohydrates, or other materials with which it is naturally found when total nucleic acid is isolated from the source cells; (2) is not be linked to all or a portion of a polynucleotide to which the nucleic acid molecule is linked in nature; (3) is operably linked to a polynucleotide which it is not linked to in nature; and/or, (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. As used herein, the term “naturally occurring” or “native” or “naturally found” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The identity of two or more nucleic acid or polypeptide molecules is determined by comparing the sequences. As known in the art, “identity” means the degree of sequence relatedness between nucleic acid molecules or polypeptides as determined by the match between the units making up the molecules (i.e., nucleotides or amino acid residues). Identity measures the percent of identical matches between the smaller of two or more sequences with gap alignments (if any) addressed by a particular mathematical model or computer program (i.e., an algorithm). Identity between nucleic acid sequences may also be determined by the ability of the related sequence to hybridize to the nucleic acid sequence or isolated nucleic acid molecule. In defining such sequences, the term “highly stringent conditions” and “moderately stringent conditions” refer to procedures that permit hybridization of nucleic acid strands whose sequences are complementary, and to exclude hybridization of significantly mismatched nucleic acids. Examples of “highly stringent conditions” for hybridization and washing are 0.015 M sodium chloride, 0.0015 M sodium citrate at 65-68° C. or 0.015 M sodium chloride, 0.0015 M sodium. citrate, and 50% formamide at 42° C. (see, for example, Sambrook, Fritsch & Maniatis, Molecular Cloning: A Laboratory Manual (2nd ed., Cold Spring Harbor Laboratory, 1989); Anderson et al., Nucleic Acid Hybridisation: A Practical Approach Ch. 4 (IRL Press Limited)). The term “moderately stringent conditions” refers to conditions under which a DNA duplex with a greater degree of base pair mismatching than could occur under “highly stringent conditions” is able to form. Exemplary moderately stringent conditions are 0.0.15 M sodium chloride, 0.0015 M sodium citrate at 50-65° C. or 0.015 M sodium chloride, 0.0015 M sodium citrate, and 20% formamide at 37-50° C. By way of example, moderately stringent conditions of 50° C. in 0.015 M sodium ion will allow about a 21% mismatch. During hybridization, other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH.

In preferred embodiments of the present invention, vectors are used to transfer a nucleic acid sequence encoding a polypeptide to a cell. A vector is any molecule used to transfer a nucleic acid sequence to a host cell. In certain cases, an expression vector is utilized. An expression vector is a nucleic acid molecule that is suitable for transformation of a host cell and contains nucleic acid sequences that direct and/or control the expression of the transferred nucleic acid sequences. Expression includes, but is not limited to, processes such as transcription, translation, and splicing, if introns are present. Expression vectors typically comprise one or more flanking sequences operably linked to a heterologous nucleic acid sequence encoding a polypeptide. Flanking sequences may be homologous (i.e., from the same species and/or strain as the host cell), heterologous (i.e., from a species other than the host cell species or strain), hybrid (i.e., a combination of flanking sequences from more than one source), or synthetic, for example.

A flanking sequence is preferably capable of effecting the replication, transcription and/or translation of the coding sequence and is operably linked to a coding sequence. As used herein, the term operably linked refers to a linkage of polynucleotide elements in a functional relationship. For instance, a promoter or enhancer is operably linked to a coding sequence if it affects the transcription of the coding sequence. However, a flanking sequence need not necessarily be contiguous with the coding sequence, so long as it functions correctly. Thus, for example, intervening untranslated yet transcribed sequences can be present between a promoter sequence and the coding sequence and the promoter sequence may still be considered operably linked to the coding sequence. Similarly, an enhancer sequence may be located upstream or downstream from the coding sequence and affect transcription of the sequence.

In certain embodiments, it is preferred that the flanking sequence is a trascriptional regulatory region that drives high-level gene expression in the target cell. The transcriptional regulatory region may comprise, for example, a promoter, enhancer, silencer, repressor element, or combinations thereof. The transcriptional regulatory region may be either constitutive, tissue-specific, cell-type specific (i.e., the region is drives higher levels of transcription in a one type of tissue or cell as compared to another), or regulatable (i.e., responsive to interaction with a compound such as tetracycline). The source of a transcriptional regulatory region may be any prokaryotic or eukaryotic organism, any vertebrate or invertebrate organism, or any plant, provided that the flanking sequence functions in a cell by causing transcription of a nucleic acid within that cell. A wide variety of transcriptional regulatory regions may be utilized in practicing the present invention.

Suitable transcriptional regulatory regions include the CMV promoter (i.e., the CMV-immediate early promoter); promoters from eukaryotic genes (i.e., the estrogen-inducible chicken ovalbumin gene, the interferon genes, the gluco-corticoid-inducible tyrosine aminotransferase gene, and the thymidine kinase gene); and the major early and late adenovirus gene promoters; the SV40 early promoter region (Bernoist and Chambon, 1981, Nature 290:304-10); the promoter contained in the 3′ long terminal repeat (LTR) of Rous sarcoma virus (RSV) (Yamamoto, et al., 1980, Cell 22:787-97); the herpes simplex virus thymidine kinase (HSV-TK) promoter, (Wagner et al., 1981, Proc. Natl. Acad. Sci. U.S.A. 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. U.S.A., 75:3727-31); or the tac promoter (DeBoer et al., 1983, Proc. Natl. Acad. Sci. U.S.A., 80:21-25). Tissue- and/or cell-type specific transcriptional control regions include, for example, the elastase I gene control region which is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region which is active in pancreatic beta cells (Hanahan, 1985, Nature 315:115-22); the immunoglobulin gene control region which is active in lymphoid cells (Grosschedl et al., 1984, Cell 38:647-58; Adames et al., 1985, Nature 318:533-38; Alexander et al., 1987, Mol. Cell. Biol., 7:1436-44); the mouse mammary tumor virus control region in testicular, breast, lymphoid and mast cells (Leder et al., 1986, Cell 45:485-95); the albumin gene control region in liver (Pinkert et al., 1987, Genes and Devel. 1:268-76); the alpha-feto-protein gene control region in liver (Krumlauf et al., 1985, Mol. Cell. Biol., 5:1639-48; Hammer et al., 1987, Science 235:53-58); the alpha 1-antitrypsin gene control region in liver (Kelsey et al., 1987, Genes and Devel. 1:161-71); the beta-globin gene control region in myeloid cells (Mogram et al., 1985, Nature 315:338-40; Kollias et al., 1986, Cell 46:89-94); the myelin basic protein gene control region in oligodendrocyte cells in the brain (Readhead et al., 1987, Cell 48:703-12); the myosin light chain-2 gene control region in skeletal muscle (Sani, 1985, Nature 314:283-86); the gonadotropic releasing hormone gene control region in the hypothalamus (Mason et al., 1986, Science 234:1372-78), and the tyrosinase promoter in melanoma cells (Hart, I. Semin Oncol 1996 Feb;23(l):154-8; Siders, et al. Cancer Gene Ther 1998 September-October;5(5):281-91), among others. Inducible promoters that are activated in the presence of a certain compound or condition such as light, heat, radiation, tetracycline, or heat shock proteins, for example, may also be utilized (see, for example, WO 00/10612). Other suitable promoters are known in the art.

As described above, enhancers may also be suitable flanking sequences. Enhancers are cis-acting elements of DNA, usually about 10-300 bp in length, that act on. the promoter to increase transcription. Enhancers are typically orientation- and position-independent, having been identified both 5′ and 3′ to controlled coding -sequences. Several enhancer sequences available from mammalian genes are known (i.e., globin, elastase, albumin, alpha-feto-protein and insulin). Similarly, the SV40 enhancer, the cytomegalovirus early promoter enhancer, the polyoma enhancer, and adenovirus enhancers are useful with eukaryotic promoter sequences. While an enhancer may be spliced into the vector at a position 5′ or 3′ to nucleic acid coding sequence, it is typically located at a site 5′ from the promoter. Other suitable enhancers are known in the art, and would be applicable to the present invention.

While preparing reagents of the present invention, cells may need to be transfected or transformed. Transfection refers to the uptake of foreign or exogenous DNA by a cell, and a cell has been transfected when the exogenous DNA has been introduced inside the cell membrane. A number of transfection techniques are well known in the art (i.e., Graham et al., 1973, Virology 52:456; Sambrook et al., Molecular Cloning, A Laboratory Manual (Cold Spring Harbor Laboratories, 1989); Davis et al., Basic Methods in Molecular Biology (Elsevier, 1986); and Chu et al., 1981, Gene 13:197). Such techniques can be used to introduce one or more exogenous DNA moieties into suitable host cells.

In certain embodiments, it is preferred that transfection of a cell results in transformation of that cell. A cell is transformed when there is a change in a characteristic of the cell, being transformed when it has been modified to contain a new nucleic acid. Following transfection, the transfected nucleic acid may recombine with that of the cell by physically integrating into a chromosome of the cell, may be maintained transiently as an episomal element without being replicated, or may replicate independently as a plasmid. A cell is stably transformed when the nucleic acid is replicated with the division of the cell.

The present invention further provides isolated immunogenic targets in polypeptide form. A polypeptide is considered isolated where it: (1-) has been separated from at least about 50 percent of polynucleotides, lipids, carbohydrates, or other materials with which it is naturally found when isolated from the source cell; (2) is not linked (by covalent or noncovalent interaction) to all or a portion of a polypeptide to which the “isolated polypeptide” is linked in nature; (3) is operably linked (by covalent or noncovalent interaction) to a polypeptide with which it is not linked in nature; or, (4) does not occur in nature. Preferably, the isolated polypeptide is substantially free from any other contaminating polypeptides or other contaminants that are found in its natural environiment that would interfere with its therapeutic, diagnostic, prophylactic or research use.

Immunogenic target polypeptides may be mature polypeptides, as defined herein, and may or may not have an amino terminal methionine residue, depending on the method by which they are prepared. Further contemplated are related polypeptides such as, for example, fragments, variants (i.e., allelic, splice), orthologs, homologues, and derivatives, for example, that possess at least one characteristic or activity (i.e., activity, antigenicity) of the immunogenic target. Also related are peptides, which refers to a series of contiguous amino acid residues having a sequence corresponding to at least a portion of the polypeptide from which its sequence is derived. In preferred embodiments, the peptide comprises about 5-10 amino acids, 10-15 amino acids, 15-20 amino acids, 20-30 amino acids, or 30-50 amino acids. In a more preferred embodiment, a peptide comprises 9-12 amino acids, suitable for presentation upon Class I MHC molecules, for example.

A fragment of a nucleic acid or polypeptide comprises a truncation of the sequence (i.e., nucleic acid or polypeptide) at the amino terminus (with or without a leader sequence) and/or the carboxy terminus. Fragments may also include variants (i.e., allelic, splice), orthologs, homologues, and other variants having one or more amino acid additions or substitutions or internal deletions as compared to the parental sequence. In preferred embodiments, truncations and/or deletions comprise about 10 amino acids, 20 amino acids, 30 amino acids, 40 amino acids, 50 amino acids, or more. The polypeptide fragments so produced will comprise about 10 amino acids, 25 amino acids, 30 amino acids, 40 amino acids, 50 amino acids, 60 amino acids, 70 amino acids, or more. Such polypeptide fragments may optionally comprise an amino terminal. methionine residue. It will be appreciated that such fragments can be used, for example, to generate antibodies or cellular immune responses to immunogenic target polypeptides.

A variant is a sequence having one or more sequence substitutions, deletions,. and/or additions as compared to the subject sequence. Variants may be naturally occurring or artificially constructed. Such variants may be prepared from the corresponding nucleic acid molecules. In preferred embodiments, the variants have from 1 to 3, or from 1 to 5, or from 1 to 10, or from 1 to 15, or from 1 to 20, or from 1 to 30, or from 1 to 40, or from 1 to 50, or more than 50 amino acid substitutions, insertions, additions and/or deletions.

An allelic variant is one of several possible naturally-occurring alternate forms of a gene occupying a given locus on a chromosome of an organism or a population of organisms. A splice variant is a polypeptide generated from one of several RNA transcript resulting from splicing of a primary transcript. An ortholog is a similar nucleic acid or polypeptide sequence from another species. For example, the mouse and human versions of an immunogenic target polypeptide may be considered orthologs of each other. A derivative of a sequence is one that is derived from a parental sequence those sequences having substitutions, additions, deletions, or chemically modified variants. Variants may also include fusion proteins, which refers to the fusion of one or more first sequences (such as a peptide) at the amino or carboxy terminus of at least one other sequence (such as a heterologous peptide). “Similarity” is a concept related to identity, except that similarity refers to a measure of relatedness which includes both identical matches and conservative substitution matches. If two polypeptide sequences have, for example, 10/20 identical amino acids, and the remainder are all non-conservative substitutions, then the percent identity and similarity would both be 50%. If in the same example, there are five more positions where there are conservative substitutions, then the percent identity remains 50%, but the percent similarity would be 75% (15/20). Therefore, in cases where there are conservative substitutions, the percent similarity between two polypeptides will be higher than the percent identity between those two polypeptides.

Substitutions may be conservative, or non-conservative, or any combination thereof. Conservative amino acid modifications to the sequence of a polypeptide (and the corresponding modifications to the encoding nucleotides) may produce polypeptides having functional and chemical characteristics similar to those of a parental polypeptide. For example, a “conservative amino acid substitution” may involve a substitution of a native amino acid residue with a non-native residue such that there is little or no effect on the size, polarity, charge, hydrophobicity, or hydrophilicity of the amino acid residue at that position and, in particlar, does not result in decreased immunogenicity. Suitable conservative amino acid substitutions are shown in Table I.

TABLE I

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of polypeptide using well-known techniques. For identifying suitable areas of the molecule that may be changed without destroying biological activity (i.e., MHC binding, immunogenicity), one skilled in the art may target areas not believed to be important for that activity. For example, when similar polypeptides with similar activities from the same species or from other species are known, one skilled in the art may compare the amino acid sequence of a polypeptide to such similar polypeptides. By performing such analyses, one can identify residues and portions of the molecules that are conserved among similar polypeptides. It will be appreciated that changes in areas of the molecule that are not conserved relative to such similar polypeptides would be less likely to adversely affect the biological activity and/or structure of a polypeptide. Similarly, the residues required for binding to MHC are known, and may be modified to improve binding. However, modifications resulting in decreased binding to MHC will not be appropriate in most situations. One skilled in the art would also know that, even in relatively conserved regions, one may substitute chemically similar amino acids for the naturally occurring residues while retaining activity. Therefore, even areas that may be important for biological activity or for structure may be subject to conservative amino acid substitutions without destroying the biological activity or without adversely affecting the polypeptide structure.

Other preferred polypeptide variants include glycosylation variants wherein the number and/or type of glycosylation sites have been altered compared to the subject amino acid sequence. In one embodiment, polypeptide variants comprise a greater or a lesser number of N-1inked glycosylation sites than the subject amino acid sequence. An N-1inked glycosylation site is characterized by the sequence Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. To affect O-linked glycosylation of a polypeptide, one would modify serine and/or threonine residues.

Additional preferred variants include cysteine variants, wherein one or more cysteine residues are deleted or substituted with another amino acid (e.g., serine) as compared to the subject amino acid sequence set. Cysteine variants are useful when polypeptides must be refolded into a biologically active conformation such as after the isolation of insoluble inclusion bodies. Cysteine variants generally have fewer cysteine residues than the native protein, and typically have an even number to minimize interactions resulting from unpaired cysteines.

In other embodiments, the isolated polypeptides of the current invention include fusion polypeptide segments that assist in purification of the polypeptides. Fusions can be made either at the amino terminus or at the carboxy terminus of the subject polypeptide. variant thereof. Fusions may be direct with no linker or adapter molecule or may be through a linker or adapter molecule. A linker or adapter molecule may be one or more amino acid residues, typically from about 20 to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derivatized according to the methods described herein. Suitable fusion segments include, among others, metal binding domains (e.g., a poly-histidine segment), immunoglobulin binding domains (i.e., Protein A, Protein G, T cell, B cell, Fc receptor, or complement protein antibody-binding domains), sugar binding domains (e.g., a maltose binding domain), and/or a “tag” domain (i.e., at least a portion of α-galactosidase, a strep tag peptide, a T7 tag peptide, a FLAG peptide, or other domains that can be purified using compounds that bind to the domain, such as monoclonal antibodies). This tag is typically fused to the polypeptide upon expression of the polypeptide, and can serve as a means for affinity purification of the sequence of interest polypeptide from the host cell. Affinity purification can be accomplished, for example, by column chromatography using antibodies against the tag as an affinity matrix. Optionally, the tag can subsequently be removed from the purified sequence of interest polypeptide by various means such as using certain peptidases for cleavage. As described below, fusions may also be made between a TA and a co-stimulatory components such as the chemokines CXC10 (IP-10), CCL7 (MCP-3), or CCL5 (RANTES), for example.

A fusion motif may enhance transport of an immunogenic target to an MHC processing compartment, such as the endoplasmic reticulum. These sequences, referred to as tranduction or transcytosis sequences, include sequences derived from HIV tat (see Kim et al. 1997 J. Immunol. 159:1666), Drosophila antennapedia (see Schutze-Redelmeier et al. 1996 J. Immunol. 157:650), or human period-1 protein (hPER1; in particular, SRRHHCRSKAKRSRHH).

In addition, the polypeptide or variant thereof may be fused to a homologous polypeptide to form a homodimer or to a heterologous polypeptide to form a heterodimer. Heterologous peptides and polypeptides include, but are not limited to: an epitope to allow for the detection and/or isolation of a fusion polypeptide; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the polypeptide or variant thereof.

In certain embodiments, it may be advantageous to combine a nucleic acid sequence encoding an immunogenic target, polypeptide, or derivative thereof with one or more co-stimulatory component(s) such as cell surface proteins, cytokines or chemokines in a composition of the present invention. The co-stimulatory component may be included in the composition as a polypeptide or as a nucleic acid encoding the polypeptide, for example. Suitable co-stimulatory molecules include, for instance, polypeptides that bind members of the CD28 family (i.e., CD28, ICOS; Hutloff, et al. Nature 1999, 397: 263-265; Peach, et al. J Exp Med 1994, 180: 2049-2058) such as the CD28 binding polypeptides B7.1 (CD80; Schwartz, 1992; Chen et al, 1992; Ellis, et al. J. Immunol., 156(8): 2700-9), B7.2 (CD86; Ellis, et al. J. Immunol., 156(8): 2700-9), B7-H1.2 (WO 02/79474); polypeptides which bind members of the integrin family (i.e., LFA-1 (CD11a/CD18); Sedwick, et al. J Immunol 1999, 162: 1367-1375; Wülfing, et al. Science 1998, 282: 2266-2269; Lub, et al. Immunol Today 1995, 16: 479-483) including members of the ICAM family (i.e., ICAM-1, -2 or -3); polypeptides which bind CD2 family members (i.e., CD2, signalling lymphocyte activation molecule (CDw150 or “SLAM”; Aversa, et al. J Immunol 1997, 158: 4036-4044)) such as CD58 (LFA-3; CD2 ligand; Davis, et al. Immunol Today 1996, 17: 177-187) or SLAM ligands (Sayos, et al. Nature 1998, 395: 462-469); polypeptides which bind heat stable antigen (HSA or CD24; Zhou, et al. Eur J Immunol 1997, 27: 2524-2528); polypeptides which bind to members of the TNF receptor (TNFR) family (i.e., 4-1BB (CD137; Vinay, et al. Semin Immunol 1998, 10: 481-489), OX40 (CD134; Weinberg, et al. Semin Immunol 1998, 10: 471-480; Higgins, et al. J Immunol 1999, 162: 486-493), and CD27 (Lens, et al. Semin Immunol 1998, 10: 491-499)) such as 4-1BBL (4-1BB ligand; Vinay, et al. Semin Immunol 1998, 10: 481-48; DeBenedette, et al. J Immunol 1997, 158: 551-559), TNFR associated factor-1 (TRAF-1; 4-1BB ligand; Saoulli, et al. J Exp Med 1998, 187: 1849-1862, Arch, et al. Mol Cell Biol 1998, 18: 558-565), TRAF-2 (4-1BB and OX40 ligand; Saoulli, et al. J Exp Med 1998, 187: 1849-1862; Oshima, et al. Int Immunol 1998, 10: 517-526, Kawamata, et al. JBiol Chem 1998, 273: 5808-5814), TRAF-3 (4-1BB and OX40 ligand; Arch, et al. Mol Cell Biol 1.998, 18: 558-565; Jang, et al. Biochem Biophys Res Commun 1998, 242: 613-20; Kawamata S, et al. J Biol Chem 1998, 273: 5808-5814), OX40L (OX40 ligand; Gramaglia, et al. J Immunol 1998, 161: 6510-6517), TRAF-5 (OX40 ligand; Arch, et al. Mol Cell Biol 1998, 18: 5.58-565; Kawamata, et al. J Biol Chem 1998, 273: 5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161:,4563-4571; Sine, et al. Hum. Gene Ther., 2001, 12: 1091-1102) may also be suitable.

One or more cytokines may also be suitable co-stimulatory components or “adjuvants”, either as polypeptides or being encoded by nucleic acids contained within the compositions of the present invention (Parmiani, et al. Immunol Lett 2000 Sep 15; 74(1): 41-4; Berzofsky, et al. Nature Immunol. 1: 209-219). Suitable cytokines include, for example, interleukin-2 (IL-2) (Rosenberg, et al. Nature Med. 4: 321-327 (1998)), IL-4, IL-7, IL-12 (reviewed by Pardoll, 1992; Harries, et al. J. Gene Med. 2000 July-August; 2(4):243-9; Rao, et al. J. Immunol. 156: 3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), or tumor necrosis factor-alpha (TNF-α).

Interferons may also be suitable cytokines for use in practicing the present invention. There are three main classes of interferon (alpha interferon (IFN-α), beta interferon (IFN-β) and gamma interferon (IFN-γ)) and at least 22 subtypes from among these. Many of these are available commercially. For instance, IFNs are commercially available as INFERGEN® (interferon alfacon-1; Intermune), Viraferon® (Schering-Plough), Roferon-A® (Roche) Wellferon® (Glaxo SmithKline), IFNα2b (Schering Canada, Pointe-Claire, Quebec), IFN beta-1b (Betaseron®; Berlex Laboratories), Avonex® (IFN beta-1a; Biogen); and Rebif®(IFN beta-1a; Serono, Pfizer), Actimmune® Interferon gamma-1b; Intermune). Preparations containing multiple IFN species in a single preparation are also available (i.e., IFN-alpha N3 or Alferon N). Variant and modified IFNs are also well-known (i.e., Maral, et al. Proc Am Soc Clin Oncol 22: page 174, 2003 (abstr 698); pegylated interferon alpha/Pegasys® (Roche); Peg Intron® (Schering Plough)). Other cytokines may also be suitable for practicing the present invention, as is known in the art.

Chemokines may also be utilized. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258). The chemokines CCL3 (MIP-1α) and CCL5 (RANTES) (Boyer, et al. Vaccine, 1999, 17 (Supp. 2): S53-S64) may also be of use in practicing the present invention. Other suitable chemokines are known in the art.

It is also known in the art that suppressive or negative regulatory immune mechanisms may be blocked, resulting in enhanced immune responses. For instance, treatment with anti-CTLA-4 (Shrikant, et al. Immunity, 1996, 14: 145-155; Sutmuller, et al. J. Exp. Med., 2001, 194: 823-832), anti-CD25 (Sutmuller, supra), anti-CD4 (Matsui, et al. J. Immunol., 1999, 163: 184-193), the fusion protein IL13Ra2-Fc (Terabe, et al. Nature Immunol., 2000, 1: 515-520), and combinations thereof (i.e., anti-CTLA-4 and anti-CD25, Sutmuller, supra) have been shown to upregulate anti-tumor immune responses and would be suitable in practicing the present invention.

Any of these components may be used alone or in combination with other agents. For instance, it has been shown that a combination of CD80, ICAM-1 and LFA-3 (“TRICOM”) may potentiate anti-cancer immune responses (Hodge, et al. Cancer Res. 59: 5800-5807 (1999). Other effective combinations include, for example, IL-12+GM-CSF (Ahlers, et al. J. Immunol., 158: 3947-3958 (1997); Iwasaki, et al. J. Immunol. 158: 4591-4601 (1997)), IL-12+GM-CSF +TNF-α (Ahlers, et al. Int. Immunol. 13: 897-908 (2001)), CD80 +IL-12 (Fruend, et al. Int. J Cancer, 85: 508-517 (2000); Rao, et al. supra), and CD86+GM-CSF+IL-12 (Iwasaki, supra). One of skill in the art would be aware of additional combinations useful in carrying out the present invention. In addition, the skilled artisan would be aware of additional reagents or methods that may be used to modulate such mechanisms. These reagents and methods, as well as others known by those of skill in the art, may be utilized in practicing the present invention.

Additional strategies for improving the efficiency of nucleic acid-based immunization may also be used including, for example, the use of self-replicating viral replicons (Caley, et al. 1999. Vaccine, 17: 3124-2135; Dubensky, et al. 2000. Mol. Med. 6: 723-732; Leitner, et al. 2000. Cancer Res. 60: 51-55), codon optimization (Liu, et al. 2000. Mol. Ther., 1: 497-500; Dubensky, supra; Huang, et al. 2001. J. Virol. 75: 4947-4951), in vivo electroporation (Widera, et al. 2000. J. Immunol. 164: 4635-3640), incorporation of CpG stimulatory motifs (Gurunathan, et al. Ann. Rev. Immunol., 2000, 18: 927-974; Leitner, supra; Cho, et al. J. Immunol. 168(10):4907-13), sequences for targeting of the endocytic or ubiquitin-processing pathways (Thomson, et al. 1998. J. Virol. 72: 2246-2252; Velders, et al. 2001. J. Immunol. 166: 5366-5373), Marek's disease virus type 1 VP22 sequences (J. Virol. 76(6):2676-82, 2002), prime-boost regimens (Gurunathan, supra; Sullivan, et al. 2000. Nature, 408: 605-609; Hanke, et al. 1998. Vaccine, 16: 439-445; Amara, et al. 2001. Science, 292: 69-74), and the use of mucosal delivery vectors such as Salmonella (Daiji, et al. 1997. Cell, 91: 765-775; Woo, et al. 2001. Vaccine, 19: 2945-2954). Other methods are known in the art, some of which are described below.

Chemotherapeutic agents, radiation, anti-angiogenic compounds, or other agents may also be utilized in treating and/or preventing cancer using immunogenic targets (Sebti, et al. Oncogene 2000 Dec. 27; 19(56):6566-73). For example, in treating metastatic breast cancer, useful chemotherapeutic agents include cyclophosphamide, doxorubicin, paclitaxel, docetaxel, navelbine, capecitabine, and mitomycin C, among others. Combination chemotherapeutic regimens have also proven effective including cyclophosphamide+methotrexate+5-fluorouracil; cyclophosphamide+doxorubicin+5-fluorouracil; or, cyclophosphamide+doxorubicin, for example. Other compounds such as prednisone, a taxane, navelbine, mitomycin C, or vinblastine have been utlized for various reasons. A majority of breast cancer patients have estrogen-receptor positive (ER+) tumors and in these patients, endocrine therapy (i.e., tamoxifen) is preferred over chemotherapy. For such patients, tamoxifen or, as a second line therapy, progestins (medroxyprogesterone acetate or megestrol acetate) are preferred. Aromatase inhibitors (i.e., aminoglutethimide and analogs thereof such as letrozole) decrease the availability of estrogen needed to maintain tumor growth and may be used as second or third line endocrine therapy in certain patients.

Other cancers may require different chemotherapeutic regimens. For example, metastatic colorectal cancer is typically treated with Camptosar (irinotecan or CPT-11), 5-fluorouracil or leucovorin, alone or in combination with one another. Proteinase and integrin inhibitors such as as the MMP inhibitors marimastate (British Biotech), COL-3 (Collagenex), Neovastat (Aetema), AG3340 (Agouron), BMS-275291 (Bristol Myers Squibb), CGS 27023A (Novartis) or the integrin inhibitors Vitaxin (Medimmune), or MED1522 (Merck KgaA) may also be suitable for use. In treating metastatic melanoma, suitable chemotherapeutic regimens may include levamisole (Quirt, et al. 1991. J. Clin. Oncol. 9: 729-725), BELD (bleomycin, vindesine, lomustine, and deacarbazine; Young, et al. 1985. Cancer, 55: 1879-81), BOLD (bleomycin, vincristine, lomustine, dacarbazine; Seigler, et al. 1980. Cancer, 46: 2346-8), DD (dacarbazine, actinomycin; Hochster, et al. Cancer Treatment Reports, 69: 39-42), or POC (procarbazine, vincristine, lomustine; Carmo-Pereira, et al. 1984. Cancer Treatment Reports, 68: 1211 -4), among others. As such, immunological targeting of immunogenic targets associated with colorectal cancer could be performed in combination with a treatment using those chemotherapeutic agents. Similarly, chemotherapeutic agents used to treat other types of cancers are well-known in the art and may be combined with the immunogenic targets described herein.

Many anti-angiogenic agents are known in the art and would be suitable for co-administration with the immunogenic target vaccines (see, for example, Timar, et al. 2001. Pathology Oncol. Res., 7(2): 85-94). Such agents include, for example, physiological agents such as growth factors (i.e., ANG-2, NK1,2,4 (HGF), transforming growth factor beta (TGF-β)), cytokines (i.e., interferons such as IFN-α0, -β, -γ, platelet factor 4 (PF-4), PR-39), proteases (i.e., cleaved AT-III, collagen XVIII fragment (Endostatin)), HmwKallikrein-d5 plasmin fragment (Angiostatin), prothrombin-F1-2, TSP-1), protease inhibitors (i.e., tissue inhibitor of metalloproteases such as TIMP-1, -2, or -3; maspin; plasminogen activator-inhibitors such as PAI-1; pigment epithelium derived factor (PEDF)), Tumstatin (available through ILEX, Inc.), antibody products (i.e., the collagen-binding antibodies HUIV26, HUI77, XL313; anti-VEGF; anti-integrin (i.e., Vitaxin, (Lxsys))), and glycosidases (i.e., heparinase-I, -III). “Chemical” or modified physiological agents known or believed to have anti-angiogenic potential include, for example, vinblastine, taxol, ketoconazole, thalidomide, dolestatin, combrestatin A, rapamycin (Guba, et al. 2002, Nature Med., 8: 128-135), CEP-7055 (available from Cephalon, Inc.), flavone acetic acid, Bay 12-9566 (Bayer Corp.), AG3340 (Agouron, Inc.), CGS 27023A (Novartis), tetracylcine derivatives (i.e., COL-3 (Collagenix, Inc.)), Neovastat (Aeterna), BMS-275291 (Bristol-Myers Squibb), low dose 5-FU, low dose methotrexate (MTX), irsofladine, radicicol, cyclosporine, captopril, celecoxib, D45152-sulphated polysaccharide, cationic protein (Protamine), cationic peptide-VEGF, Suramin (polysulphonated napthyl urea), compounds that interfere with the function or production of VEGF (i.e., SU5416 or SU6668 (Sugen), PTK787/ZK22584 (Novartis)), Distamycin A, Angiozyme (ribozyme), isoflavinoids, staurosporine derivatives, genistein, EMD121974 (Merck KcgaA), tyrphostins, isoquinolones, retinoic acid, carboxyamidotriazole, TNP-470, octreotide, 2-methoxyestradiol, aminosterols (i.e., squalamine), glutathione analogues (i.e., N-acteyl-L-cysteine), combretastatin A-4 (Oxigene), Eph receptor blocking agents (Nature, 414:933-938, 2001), Rh-Angiostatin, Rh-Endostatin (WO 01/93897), cyclic-RGD peptide, accutin-disintegrin, benzodiazepenes, humanized anti-avb3 Ab, Rh-PAI-2, amiloride, p-amidobenzamidine, anti-uPA ab, anti-uPAR Ab, L-phanylalanin-N-methylamides (i.e., Batimistat, Marimastat), AG3340, and minocycline. Many other suitable agents are known in the art and would suffice in practicing the present invention.

The present invention may also be utilized in combination with “non-traditional” methods of treating cancer. For example, it has recently been demonstrated that administration of certain anaerobic bacteria may assist in slowing tumor growth. In one study, Clostridium novyi was modified to eliminate a toxin gene carried on a phage episome and administered to mice with colorectal tumors (Dang, et al. P.N.A.S. USA, 98(26): 15155-15160, 2001). In combination with chemotherapy, the treatment was shown to cause tumor necrosis in the animals. The reagents and methodologies described in this application may be combined with such treatment methodologies.

Nucleic acids encoding immunogenic targets may be administered to patients by any of several available techniques. Various viral vectors that have been successfully utilized for introducing a nucleic acid to a host include retrovirus, adenovirus, adeno-associated virus (AAV), herpes virus, and poxvirus, among others. It is understood in the art that many such viral vectors are available in the art. The vectors of the present invention may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

Preferred retroviral vectors are derivatives of lentivirus as well as derivatives of murine or avian retroviruses. Examples of suitable retroviral vectors include, for example, Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of retroviral vectors can incorporate multiple exogenous nucleic acid sequences. As recombinant retroviruses are defective, they require assistance in order to produce infectious vector particles. This assistance can be provided by, for example, helper cell lines encoding retrovirus structural genes. Suitable helper cell lines include Ψ2, PA317 and PA12, among others. The vector virions produced using such cell lines may then be used to infect a tissue cell line, such as NIH 3T3 cells, to produce large quantities of chimeric retroviral virions. Retroviral vectors may be administered by traditional methods (i.e., injection) or by implantation of a “producer cell line” in proximity to the target cell population (Culver, K., et al., 1994, Hum. Gene Ther., 5 (3): 343-79; Culver, K., et al., Cold Spring Harb. Symp. Quant. Biol., 59: 685-90); Oldfield, E., .1993, Hum. Gene Ther., 4 (1): 39-69). The producer cell line is engineered to produce a viral vector and releases viral particles in the vicinity of the target cell. A portion of the released viral particles contact the target cells and infect those cells, thus delivering a nucleic acid of the present invention to the target cell. Following infection of the target cell, expression of the nucleic acid of the vector occurs.

Adenoviral vectors have proven especially useful for gene transfer into eukaryotic cells (Rosenfeld, M., et al., 1991, Science, 252 (5004): 431-4; Crystal, R., et al., 1994, Nat. Genet., 8 (1): 42-51), the study eukaryotic gene expression (Levrero, M., et al., 1991, Gene, 101 (2): 195-202), vaccine development (Graham, F. and Prevec, L., 1992, Biotechnology, 20: 363-90), and in animal models (Stratford-Perricaudet, L., et al., 1992, Bone Marrow Transplant., 9 (Suppl. 1): 151-2 ; Rich, D., et al., 1993, Hum. Gene Ther., 4 (4): 461-76). Experimental routes for administrating recombinant Ad to different tissues in vivo have included intratracheal instillation (Rosenfeld, M., et al, 1992, Cell, 68 (1): 143-55) injection into muscle (Quantin, B., et al., 1992, Proc. Natl. Acad. Sci. U.S.A., 89 (7): 2581-4), peripheral intravenous injection (Herz, J., and Gerard, R., 1993, Proc. Natl. Acad. Sci. U.S.A., 90 (7): 2812-6) and stereotactic inoculation to brain (Le Gal La Salle, G., et al., 1993, Science, 259 (5097): 988-90), among others.

Adeno-associated virus (AAV) demonstrates high-level infectivity, broad host range and specificity in integrating into the host-cell genome (Hermonat, P., et al., 1984, Proc. Natl. Acad. Sci. U.S.A., 81 (20): 6466-70). And Herpes Simplex Virus type-1 (HSV-1) is yet another attractive vector system, especially for use in the nervous system because of its neurotropic property (Geller, A., et al., 1991, Trends Neurosci., 14 (10): 428-32; Glorioso, et al., 1995, Mol. Biotechnol., 4 (1): 87-99; Glorioso, et al., 1995, Annu. Rev. Microbiol., 49: 675-710).

Poxvirus is another useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). Poxviruses shown to be useful include vaccinia, NYVAC, avipox, fowlpox, canarypox, ALVAC, and ALVAC(2), among others.

NYVAC (vP866) was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R); hemorrhagic region (u; B13R+B14R); A type inclusion body region (ATI; A26L); hemagglutinin gene (HA; A56R); host range gene region (C7L-K1L); and, large subunit, ribonucleotide reductase (14L). NYVAC is a genetically engineered vaccinia virus strain that was generated by the specific deletion of eighteen open reading frames encoding gene products associated with virulence and host range. NYVAC has been show to be useful for expressing TAs (see, for example, U.S. Pat. No. 6,265,189). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively.

ALVAC-based recombinant viruses (i.e., ALVAC-1 and ALVAC-2) are also suitable for use in practicing the present invention (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

Another useful poxvirus vector is TROVAC. TROVAC refers to an attenuated fowlpox that was a plaque-cloned isolate derived from the FP-1 vaccine strain of fowlpoxvirus which is licensed for vaccination of 1 day old chicks. TROVAC was likewise deposited under the terms of the Budapest Treaty with the ATCC, accession number 2553.

“Non-viral” plasmid vectors may also be suitable in practicing the present invention. Preferred plasmid vectors are compatible with bacterial, insect, and/or mammalian host cells. Such vectors include, for example, PCR-II, pCR3, and pcDNA3.1 (Invitrogen, San Diego, Calif.), pBSII (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (PCT pub. No. WO 90/14363) and pFastBacDual (Gibco-BRL, Grand Island, N.Y.) as well as Bluescript® plasmid derivatives (a high copy number COLE1-based phagemid, Stratagene Cloning Systems, La Jolla, Calif.), PCR cloning plasmids designed for cloning Taq-amplified PCR products (e.g., TOPO™ TA cloning® kit, PCR2.1® plasmid derivatives, Invitrogen, Carlsbad, Calif.). Bacterial vectors may also be used with the current invention. These vectors include, for example, Shigella, Salmonella, Vibrio cholerae, Lactobacillus, Bacille calmette guérin (BCG), and Streptococcus (see for example, WO 88/6626; WO 90/0594; WO 91/13157; WO 92/1796; and WO 92/21376). Many other non-viral plasmid expression vectors and systems are known in the art and could be used with the current invention.

Suitable nucleic acid delivery techniques include DNA-ligand complexes, adenovirus-ligand-DNA complexes, direct injection of DNA, CaPO₄ precipitation, gene gun techniques, electroporation, and colloidal dispersion systems, among others. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6: 77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

An immunogenic target may also be administered in combination with one or more adjuvants to boost the immune response. Exemplary adjuvants are shown in Table II below:

TABLE II
Types of Immunologic Adjuvants

| Type of | | |
|---|---|---|
| Adjuvant | General Examples | Specific Examples/References |
| Gel-type | Aluminum hydroxide/ | (Aggerbeck and Heron, 1995) |
| | phosphate (“alum adjuvants”) | |
| | Calcium phosphate | (Relyveld, 1986) |
| Microbial | Muramyl dipeptide (MDP) | (Chedid et al., 1986) |
| | Bacterial exotoxins | Cholera toxin (CT), E.coli labile toxin |
| | | (LT) (Freytag and Clements, 1999) |
| | Endotoxin-based adjuvants | Monophosphoryl lipid A (MPL) |
| | | (Ulrich and Myers, 1995) |
| | Other bacterial | CpG oligonucleotides (Corral and |
| | | Petray, 2000), BCG sequences (Krieg, et |
| | | al. Nature, 374: 576), tetanus toxoid |
| | | (Rice, et al. J. Immunol., 2001, 167: |
| | | 1558-1565) |
| Particulate | Biodegradable | (Gupta et al., 1998) |
| | Polymer microspheres | |
| | Immunostimulatory complexes | (Morein and Bengtsson, 1999) |
| | (ISCOMs) | |
| | Liposomes | (Wassef et al., 1994) |
| Oil-emulsion | Freund's incomplete adjuvant | (Jensen et al., 1998) |
| and | Microfluidized emulsions | MF59 (Ott et al., 1995) |
| surfactant- | | SAF (Allison and Byars, 1992) |
| based | | (Allison, 1999) |
| adjuvants | Saponins | QS-21 (Kensil, 1996) |
| Synthetic | Muramyl peptide derivatives | Murabutide (Lederer, 1986) |
| | | Threony-MDP (Allison, 1997) |
| | Nonionic block copolymers | L121 (Allison, 1999) |
| | Polyphosphazene (PCPP) | (Payne et al., 1995) |
| | Synthetic polynucleotides | Poly A: U, Poly I: C (Johnson, 1994) |
| | Thalidomide derivatives | CC-4047/ACTIMID (J. Immunol., |
| | | 168(10): 4914-9) |

The immunogenic targets of the present invention may also be used to generate antibodies for use in screening assays or for immunotherapy. Other uses would be apparent to one of skill in the art. The term “antibody” includes antibody fragments, as are known in the art, including Fab, Fab₂, single chain antibodies (Fv for example), humanized antibodies, chimeric antibodies, human antibodies, produced by several methods as are known in the art. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boemer et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). The antibodies or derivatives therefrom may also be conjugated to therapeutic moieties such as cytotoxic drugs or toxins, or active fragments thereof such as diptheria A chain, exotoxin A chain, ricin A chain, abrin A chain, curcin, crotin, phenomycin, enomycin, among others. Cytotoxic agents may also include radiochemicals. Antibodies and their derivatives may be incorporated into compositions of the invention for use in vitro or in vivo.

Nucleic acids, proteins, or derivatives thereof representing an immunogenic target may be used in assays to determine the presence of a disease state in a patient, to predict prognosis, or to determine the effectiveness of a chemotherapeutic or other treatment regimen. Expression profiles, performed as is known in the art, may be used to determine the relative level of expression of the immunogenic target. The level of expression may then be correlated with base levels to determine whether a particular disease is present within the patient, the patient's prognosis, or whether a particular treatment regimen is effective. For example, if the patient is being treated with a particular chemotherapeutic regimen, an decreased level of expression of an immunogenic target in the patient's tissues (i.e., in peripheral blood) may indicate the regimen is decreasing the cancer load in that host. Similarly, if the level of expresssion is increasing, another therapeutic modality may need to be utilized. In one embodiment, nucleic acid probes corresponding to a nucleic acid encoding an immunogenic target may be attached to a biochip, as is known in the art, for the detection and quantification of expression in the host.

It is also possible to use nucleic acids, proteins, derivatives therefrom, or antibodies thereto as reagents in drug screening assays. The reagents may be used to ascertain the effect of a drug candidate on the expression of the immunogenic target in a cell line, or a cell or tissue of a patient. The expression profiling technique may be combined with high throughput screening techniques to allow rapid identification of useful compounds and monitor the effectiveness of treatment with a drug candidate (see, for example, Zlokamik, et al., Science 279, 84-8 (1998)). Drug candidates may be chemical compounds, nucleic acids, proteins, antibodies, or derivatives therefrom, whether naturally occurring or synthetically derived. Drug candidates thus identified may be utilized, among other uses, as pharmaceutical compositions for administration to patients or for use in further screening assays.

Administration of a composition of the present invention to a host may be accomplished using any of a variety of techniques known to those of skill in the art. The composition(s) may be processed in accordance with conventional methods of pharmacy to produce medicinal agents for administration to patients, including humans and other mammals (i.e., a “pharmaceutical composition”). The pharmaceutical composition is preferably made in the form of a dosage unit containing a given amount of DNA, viral vector particles, polypeptide or peptide, for example. A suitable daily dose for a human or other mammal may vary widely depending on the condition of the patient and other. factors, but, once again, can be determined using routine methods.

The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a nucleic acid or polypeptide used to induce or enhance an effective immune response. It is preferred that compositions of the present invention provide for the induction or enhancement of an anti-tumor immune response in a host which protects the host from the development of a tumor and/or allows the host to eliminate an existing tumor from the body.

For oral administration, the pharmaceutical composition may be of any of several forms including, for example, a capsule, a tablet, a suspension, or liquid, among others. Liquids may be administered by injection as a composition with suitable carriers including saline, dextrose, or water. The term parenteral as used herein includes subcutaneous, intravenous, intramuscular, intrasternal, infusion, or intraperitoneal administration. Suppositories for rectal administration of the drug can be prepared by mixing the drug with a suitable non-irritating excipient such as cocoa butter and polyethylene glycols that are solid at ordinary temperatures but liquid at the rectal temperature.

The dosage regimen for immunizing a host or otherwise treating a disorder or a disease with a composition of this invention is based on a variety of factors, including the type of disease, the age, weight, sex, medical condition of the patient, the severity of the condition, the route of administration, and the particular compound employed. For example, a poxviral vector may be administered as a composition comprising 1×10⁶ infectious particles per dose. Thus, the dosage regimen may vary widely, but can be determined routinely using standard methods.

In certain embodiments, cytokines may be. administered in what would be considered by those of skill in the art to be “high doses”. For example, a cytokine such as IFN may be administered to a patient repeatedly (i.e. daily for 2, 3, 4, 5, 6 or 7 days/week) over one or more weeks or months. The dose may also be given once daily, or more than once a day. In one embodiment, IFNα2b (Schering Canada, Pointe-Claire, Quebec) may be administered using the dosages set forth by Kirkwood, et al. (J.Clin.Oncol. 14: 7-17, 1996; 20 MU/m²/d IV 5 days/week×4 weeks). Dosages may be discontinued and restarted as necessary. For instance, IFNα2b dose could be discontinued and then restarted at a dose reduction if severe toxicity (grade 3 or 4, defined by the common toxicity criteria established by the National Cancer Institute Cancer Treatment Evaluation Program; Kirkwood, et al. 2001. J.Clin.Oncol. 19, 2370-2380) is observed. Subsequent decreases may also be made in; some patients for recurrent severe toxicity.

A prime-boost regimen may also be utilized (WO 01/30382 A1) in which the targeted immunogen is initially administered in a priming step in one form followed by a boosting step in which the targeted immunogen is administered in another form. The form of the targeted immunogen in the priming and boosting steps are different. For instance, if the priming step utilized a nucleic acid, the boost may be administered as a peptide. Similarly, where a priming step utilized one type of recombinant virus (i.e., ALVAC), the boost step may utilize another type of virus (i.e., NYVAC). This prime-boost method of administration has been shown to induce strong immunological responses.

While the compositions of the invention can be administered as the sole active pharmaceutical agent, they can also be used in combination with one or more other compositions or agents (i.e., other immunogenic targets, co-stimulatory molecules, adjuvants). When administered as a combination, the individual components can be formulated as separate compositions administered at the same time or different times, or the components can be combined as a single composition.

Injectable preparations, such as sterile injectable aqueous or oleaginous suspensions, may be formulated according to known methods using suitable dispersing or. wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. For instance, a viral vector such as a poxvirus may be prepared in 0.4% NaCl. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

For topical administration, a suitable topical dose of a composition may be administered one to four, and preferably two or three times daily. The dose may also be administered with intervening days during which no does is applied. Suitable compositions may comprise from 0.001% to 10% w/w, for example, from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes) and drops suitable for administration to the eye, ear, or nose.

The pharmaceutical compositions may also be prepared in a solid form (including granules, powders or suppositories). The pharmaceutical compositions may be subjected to conventional pharmaceutical operations such as sterilization and/or may contain conventional adjuvants, such as preservatives, stabilizers, wetting agents, emulsifiers, buffers etc. Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

Pharmaceutical compositions comprising a nucleic acid or polypeptide of the present invention may take any of several forms and may be administered by any of several routes. In preferred embodiments, the compositions are administered via a parenteral route (intradermal, intramuscular or subcutaneous) to induce an immune response in the host. Alternatively, the composition may be administered directly into a lymph node (intranodal) or tumor mass (i.e., intratumoral administration). For example, the dose could be administered subcutaneously at days 0, 7, and 14. Suitable methods for immunization using compositions comprising TAs are known in the art, as shown for p53 (Hollstein et al., 1991), p21-ras (Almoguera et al., 1988), HER-2 (Fendly et al., 1990), the melanoma-associated antigens (MAGE-1; MAGE-2) (van der Bruggen et al., 1991), p97 (Hu et al., 1988), melanoma-associated antigen E (WO 99/30737) and carcinoembryonic antigen (CEA) (Kantor et al., 1993; Fishbein et al., 1992; Kaufman et al., 1991), among others.

Preferred embodiments of administratable compositions include, for example, nucleic acids or polypeptides in liquid preparations such as suspensions, syrups, or elixirs. Preferred injectable preparations include, for example, nucleic acids or polypeptides suitable for parental, subcutaneous, intradermal, intramuscular or intravenous administration such as sterile suspensions or emulsions. For example, a recombinant poxvirus may be in admixture with a suitable carrier, diluent, or excipient such as sterile water, physiological saline, glucose or the like. The composition may also be provided in lyophilized form for reconstituting, for instance, in isotonic aqueous, saline buffer. In addition, the compositions can be co-administered or sequentially administered with other antineoplastic, anti-tumor or anti-cancer agents and/or with agents which reduce or alleviate ill effects of antineoplastic, anti-tumor or anti-cancer agents.

A kit comprising a composition of the present invention is also provided. The kit can include a separate container containing a suitable carrier, diluent or excipient. The kit can also include an additional anti-cancer, anti-tumor or antineoplastic agent and/or an agent that reduces or alleviates ill effects of antineoplastic, anti-tumor or anti-cancer agents for co- or sequential-administration. Additionally, the kit can include instructions for mixing or combining ingredients and/or administration.

A-better understanding of the present invention and of its many advantages will be had from the following examples, given by way of illustration.

### EXAMPLES

### Example 1

### Materials and Methods

Patients: For entry into this study, patients were required to have histologically confirmed malignant melanoma at high risk of developing metastases (pT3 or higher, any N, any M by AJCC staging), have the HLA-A*0201 haplotype, be older than 18 with an ECOG performance status of 0 or 1, and give informed, written consent according to national and institutional guidelines before treatment. All patients in this trial had completed a vaccination protocol as part of a phase I trial sponsored by Aventis-Pasteur. The vaccine protocol involved injections of the ALVAC(2)-gp100M recombinant virus (an investigational product of Aventis-Pasteur made from a second generation canarypox virus expressing a full length gp100 gene encoding two epitopes modified for enhanced HLA class I binding) along with the two modified peptide epitopes. (Van der Burg, et al. 2002. Clin.Cancer Res. 8, 1019-1027 (2002); Marshall, et al. 2000. J.Clin.Oncol. 18, 3964-3973).

Treatment with HDI: IFNγ2b (Schering Canada, Pointe-Claire, Quebec) was administered using the dose and schedule previously tested. (Kirkwood,et al. 1996. J.Clin.Oncol. 14, 7-17) HDI consisted of 20 MU/m²/d IV 5 days/week×4 weeks. The IFNα2b dose was held and then restarted at a 33% dose reduction if severe toxicity (grade 3 or 4, defined by the common toxicity criteria established by the National Cancer Institute Cancer Treatment Evaluation Program; Kirkwood, et al. 2001. J.Clin.Oncol. 19, 2370-2380) was observed. A second decrease of 33% of the original dosage was made in some patients for recurrent severe toxicity.

Study design: After being discharged from the Aventis-sponsored vaccine trial, but still considered to be at high-risk for developing metastatic disease, patients were administered HDI after giving informed consent. Patients were monitored for toxicity weekly during the first month of study (while on HDI) and then for toxicity and disease status at monthly intervals for 3 months. Radiologic evaluation was performed at 3 months of follow-up to assess tumour response. Peripheral blood was collected for immunological monitoring at each time point in sodium heparin containing tubes. Peripheral blood mononuclear cells (PBMC) were isolated by density gradient centrifugation using Ficoll-Hypaque 1.077 (Amersham Pharmacia, Sweden). Cells were washed twice in phosphate buffered saline (PBS) and frozen in 10% DMSO and 90% autologous heat-inactivated plasma (56° C. for 30 min). Aliquoted cells were kept in liquid nitrogen until use.

Reagents: Peptides: Peptides (provided by Aventis Pasteur, Toronto, Canada) corresponded to the influenza (FLU) matrix protein MP, residues 58-66, (GILGFVFTL; Gotch,F., et al. 1987. Nature 326, 881-882), two dominant epitopes from the melanoma antigen, gp100, modified to increase binding to class I MHC, gp100:209-2M (IMDQVPFSV) and gp100:280-9V (YLEPGPVTV; Parkhurst, M. R., et al. 1996. J.Immunol. 157, 2539-2548) and the HIV p17 Gag protein derived peptide (SLYNTVATL; Parker, K. C., et al. 1992. J.Immunol. 149, 3580-3587). All peptides were HLA-A*0201 restricted CTL epitopes. The gp100 peptides were dissolved in water (5 mg/ml stock solution) and the others were dissolved in DMSO (Sigma, St. Louis, Mo.) (10 mg/ml stock solution). Antibodies and tetramers: CD8-FITC antibodies were purchased from PharMingen (San Francisco, Calif.). BB7.2 (anti-HLA-A2; Parham, et al. 1981. Hum.Immunol. 3, 277-299) hybridomas were obtained from the American Type Culture Collection (ATCC) (Vanassa, Va.). Antibodies from this hybridoma were purified, and labeled with FITC, in our laboratory. Three purified, soluble recombinant HLA-A*0201-peptide complexes bound to phycoerythrin (PE)-labeled streptavidin were purchased from ProImmune Ltd. (Oxford, UK). The peptide sequences were YLEPGPVTV (Lot No. BL/0757), IMDQVPFSV (Lot No. BL/0755), and GILGFVFTL (Lot No. BL/0839). Cell Lines. Human lymphoblastoid T2 cells were obtained from the ATCC. Deletion of the TAP-transporter gene locus in T2 cells prevents delivery of cytoplasmic peptides into the ER. As a result, surface HLA expression is defective but can be rescued with exogenous peptides. The large number of identical peptide-MHC complexes on peptide loaded T2 cells make them potent antigen presenting cells (APCs).

HLA-typing: HLA-A2⁺ patients were identified using flow cytometry and BB7.2 antibodies. Molecular subtyping of HLA-A2 was performed by the HLA-laboratory at Aventis-Pasteur, using sequence-specific primer-PCR.

Short term in vitro stimulation of PBMC: Cryopreserved PBMC were thawed, washed, and incubated overnight in AIM-V medium (Gibco, Burlington, Ontario) at 37° C. in 5% CO₂. Cells were counted the next day in a hemocytometer and 1 ml of a cell suspension, adjusted to 2-3×10⁶ cells/ml in AIM-V plus 10% AB serum (Sigma, St. Louis, Mo.) (complete media, CM), was plated in single wells of a 24 well polystyrene tissue-culture grade plate (Becton Dickinson Labware, Franklin Lakes, N.J.). The cultures were then either stimulated with the gp100 peptides (gp100:209-2M and gp100:280-9V, together), at a final concentration of 25 μg/ml or with MP58-66 peptides added at a final concentration of 10 μg/ml. IL-2 (50 IU/ml) (Chiron, Emeryville, Calif.) was added on days 3 and 6 after peptide stimulation. At the end of the 8-9 day culture period, cells were harvested and washed before further testing.

ELISPOT assays: HA-multiscreen plates (Millipore, Bedford, Mass.) were coated overnight at room temperature with 75 μl of anti-IFN-γ mAb from the 1-DIK clone (MABTECH, Stockholm, Sweden) (2 μg/ml in PBS). The plates were then washed with PBS, to remove unbound antibody, and blocked with 0.5% BSA/PBS for 1 h at room temperature. PBMC were added in duplicate or triplicate wells in the presence or absence of peptide. The two modified gp100 peptides were added at a final concentration of 25 μg/ml and the FLU peptides were added at a final concentration of 10 μg/ml. Mitogenic stimulation was performed with phorbol myristic acetate (PMA) (20 ng/ml) (Sigma) and lonomycin (1 μg/ml)(Calbiochem, San Diego, Calif.). IL-2 (100 IU/ml) was included in all cultures unless stimulated by mitogens. After incubation at 37° C. in 5% CO₂ for 24 h, the cells were discarded, and the plates were washed extensively with 0.05% Tween/PBS. Secondary biotinylated anti-IFN-γ mAbs (clone 7-B6-1, MABTECH) were then added (75 μl/well at 1 μg/ml) and left for 2 h at room temperature, followed by extraavidin-conjugated alkaline phosphatase (Sigma) for an additional 1 h. The plates were developed using NBT/BCIP phosphatase substrate solution (Sigma) and counted using a stereomicroscope at 40× and an automated ELISPOT reader (Carl Zeiss Vision, Germany). Statistical analysis was carried out using Microsoft Excel software.

Chromium release assays for cellular cytotoxicity: T2 tumor targets in exponential growth phase were collected by centrifugation and incubated with 2 μg/ml of peptides (g209M and g280V, mixed 1:1, or FLU peptide) for 2 h at 37° C. and then washed twice to remove free peptide. The cells were then resuspended in two drops of 100% fetal calf serum, and radiolabeled with 50 μl of sodium chromate (7.14 mCi/ml) (Dupont, NEN, Boston, Mass.) for 1 h. Effector cells, purified from the 8 day peptide stimulated cultures by density centrifligation over Ficoll-Hypaque columns, were added at varying effector: target ratios in 100 μl of CM to individual wells of a U-bottom plate. Chromium labeled targets were washed 3 times with α-MEM+l% FCS and 100 μl of target cells (2×10⁴/ml in CM) were added to each well. The plates were centrifuged at 600 rpm for 3 min and then incubated at 37° C. for 4 h. Plates were then centrifuged at 800 rpm for 5 min and 100 μl of the supernatant transferred to Fisherbrand flint glass tubes (Fisher Scientific, Pittsburgh, Pa.) and counted in a γ-counter (CompuGamma Model 1282, LKB, Stockholm, Sweden). Total release (TR) was measured by lysis of tumor targets with 1% acetic acid and spontaneous release (SR) was measured in the absence of effector cells. Percent cytotoxicity was determined by the ratio (cpm-SR)/(TR-SR)×100%.

Immunofluorescence: Cell staining was performed as previously described using cells taken at the end of the in vitro culture period. (Spaner, et al. 1998. J. Immunol. 160, 2655-2664).

### Example 2

### Treatment of Melanoma Using a High-Dose IFN-α and a Recombinant Viral Vector

Toxicity: As shown in Table 1, seven HLA-A*0201⁺ patients received one month of high dose IFNα2b (Schering Canada, Pointe-Claire, Quebec) (HDI) between 1.5 months and 17 months (mean=7.2±4.9 S.D.) after their last injection of a vaccine containing gp100 and its known HLA-A*0201 binding epitopes (Parkhurst, 1996, supra; Bakker, et al. 1997. Int.J.Cancer 70, 302-309). All 7 patients completed the course of HDI and no evidence of disease progression was noted. In fact, two patients (Ml66 and M335) developed marked disease reduction after HDI and their clinical course will be described in greater detail below. Patients developed typical toxicities associated with HDI including flu-like symptoms, cytopenias, and liver function test abnormalities, which lasted only during the time of HDI (Table 2). One patient (M160) developed neuro-psychiatric symptoms, requiring the institution of anti-depressants, which also cleared within a week of stopping HDI. One patient (M335) developed vitiligo around skin deposits of melanoma (described below). Dose reductions and treatment delays due to toxicity were experienced by all 7 patients (Table 2) which is somewhat higher than the 33% incidence reported for 396 patients in the E1694 Intergroup trial. (Kirkwood, 2002, supra)

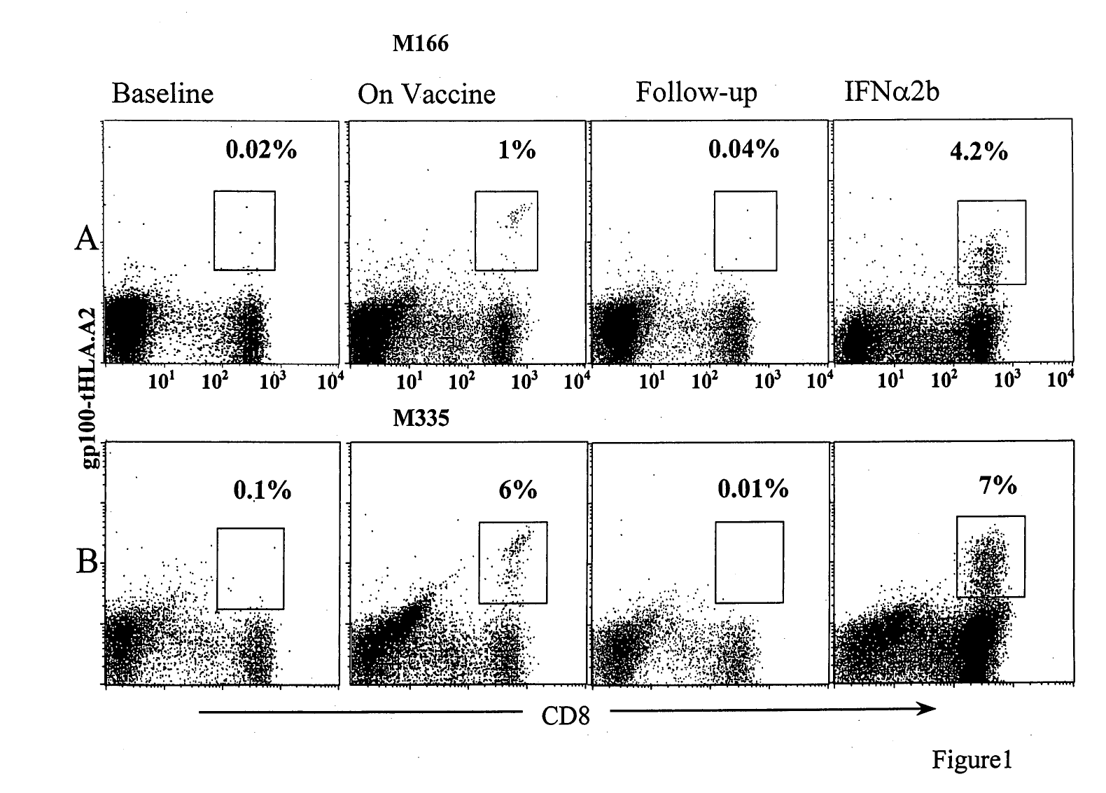

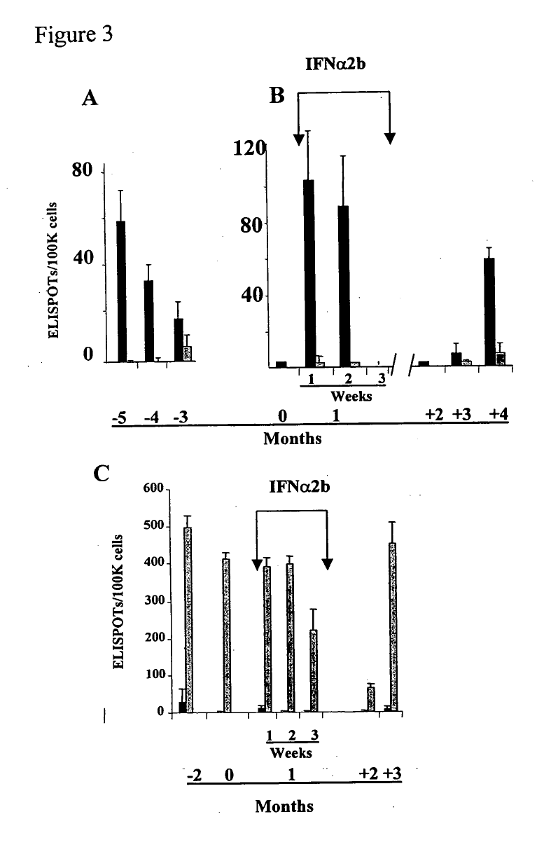

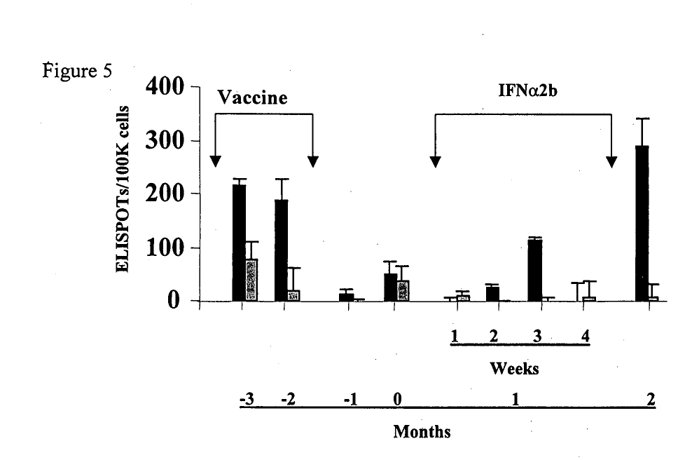

Recall of vaccine-induced anti-gp100 T cell responses by HDI: The design of this study, which used HLA-A*0201⁺ patients previously immunized with gp100 based vaccines, made it relatively easy to monitor the immunological events associated with the subsequent administration of HDI. Tumor-reactive T cells could be enumerated in ELISPOT assays (Pass, et al. 1998. Cancer J. Sci. Am., 4: 316-323; Scheibenbogen, et al. 1997. Int.J.Cancer 71, 932-936). ELISPOT assays determine the frequency of T cells that secrete IFN-γ after stimulation by the two immunogenic HLA-A*-0201 binding gp100 peptides (gp100:209-2M and gp100:g280-9V). Flow cytometric assays using tetramers of recombinant HLA-A*0201 folded around the gp100 peptides (Klenerman, et al. 2002. Nat. Rev. Immunol. 2: 263-272) were also performed. None of the patients had evidence of circulating gp100-reactive T cells by any of these two assays before beginning the month of HDI (FIGS. 1a and b, “Follow-up” dot-plots; FIG. 3b; FIG. 5; and data not shown). However, 4/7 patients had a measurable increase in the frequency of gp100-reactive T cells (arbitrarily set at >1/10⁴ cells) at some point during the vaccination protocol (Table 1, column 7 and FIG. 1, “On Vaccine” dot-plots), although this increase was only transient (FIG. 1, “Follow-up” dot-plots; FIG. 3b; FIG. 5; and data not shown). In these patients, measurable frequencies of gp100-reactive T cells again developed by the second week of HDI (Table I column 7; FIG. 1, “IFN-α2b” dot-plots; FIG. 3b; FIG. 5). However, if patients had not achieved a measurable anti-gp100 response to vaccination, treatment with HDI did not lead to a measurable increase in gp100-reactive T cells (Table 1, column 7, patients M126, M246, and M260). As a control to ensure that the failure to demonstrate gp100-reactive T cells in these patients was not due to technical difficulties associated with the cryopreservation and culture conditions, the response to the HLA-A*0201 binding peptide, influenza (FLU) matrix protein MP, residues 58-66, (GILGFVFTL), was measured at the same time using IFN-γ ELISPOT assays and peptide-folded tetramers (FIG. 3c). It is known that 60-70% of patients have memory T cell responses to FLU from previous natural infections with this virus. In all cases, the culture conditions were sufficient to support the development of FLU-reactive T cells (FIG. 3c and data not shown), suggesting that the absence of gp100-reactive T cells in the blood of these patients was real. Interestingly the FLU-responses did not always increase when the patients were treated with HDI compared to the baseline values (FIG. 3c and data not shown).

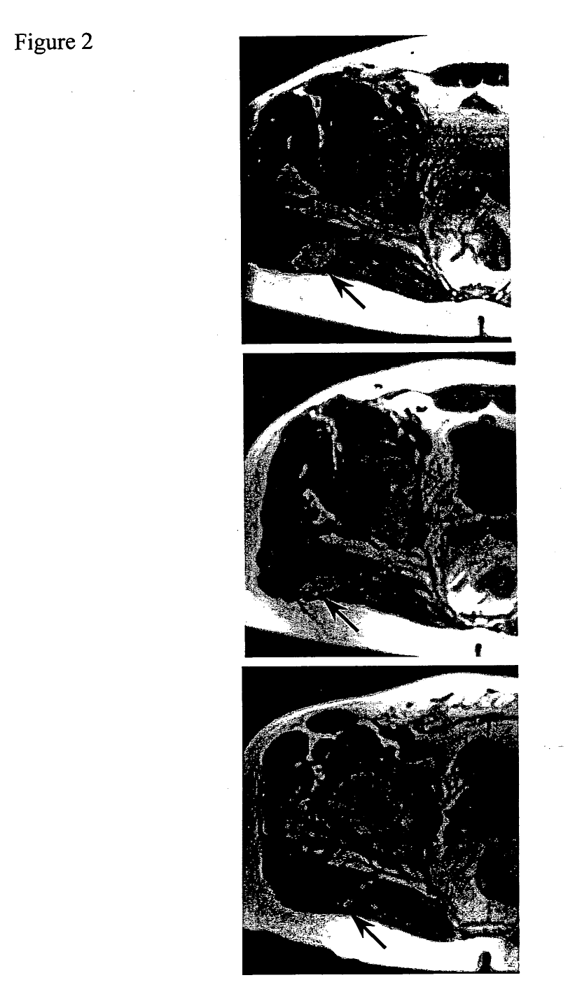

Association of increased gp100-reactive T cells after HDI and clinical responses in M166: One patient (M166) was a 31 year old male who initially presented with a 0.6 mm deep melanoma in his neck. Six years later, he developed a small bowel obstruction from a mesenteric metastatic melanoma deposit that was resected surgically. No other metastatic disease was evident until he was considered for the melanoma vaccine study 18 months later and found to have a mass in the gluteal region (FIG. 2a, arrow). A clinical decision was made to observe the mass during the vaccination period because of the difficult nature of the surgery required for its resection. Three months after completing active vaccination, the mass was somewhat smaller (FIG. 2b, arrow). The patient received HDI 3 months after that, the mass subsequently disappeared, and has not recurred as of 8 months later, at the time of the last follow-up visit (FIG. 2c, arrow).

As shown in FIG. 1a and FIG. 3a, M166 mounted an immune response to the gp100-based vaccines. During vaccination, gp100-reactive CD8⁺ T cells comprised 1% of the total cells in an 8 day culture of PBMC primed with gp100:109-2M and gp100:g280-9V as measured by tetramer staining and flow cytometric analysis (FIG. 1a, “On Vaccine” dot-plot). At the end of the vaccination period, the frequency of gp100-reactive T cells fell (FIG. 3A) and disappeared by the time that HDI was instituted (FIG. 1a, “Follow-up” dot-plot; FIG. 3b). However, after one week of HDI, the frequency of IFN-γ producing gp100-reactive T cells increased to ˜1/1000 (FIG. 3b) and the number of CD8⁺ T cells that were stained by the tetramers of HLA-A*0201 and the gp100 peptides was 4.2% of the cells in the culture (FIG. 1a, “IFNα2b” dot-plot). Although the frequency of gp100-reactive T cells in the ELISPOT assay varied, it was still elevated 4 months after completing HDI (FIG. 3b). In this patient, FLU-reactive CD8⁺ T cell frequencies were relatively constant despite HDI and the changing gp100-reactive T cell frequencies (FIG. 3c).

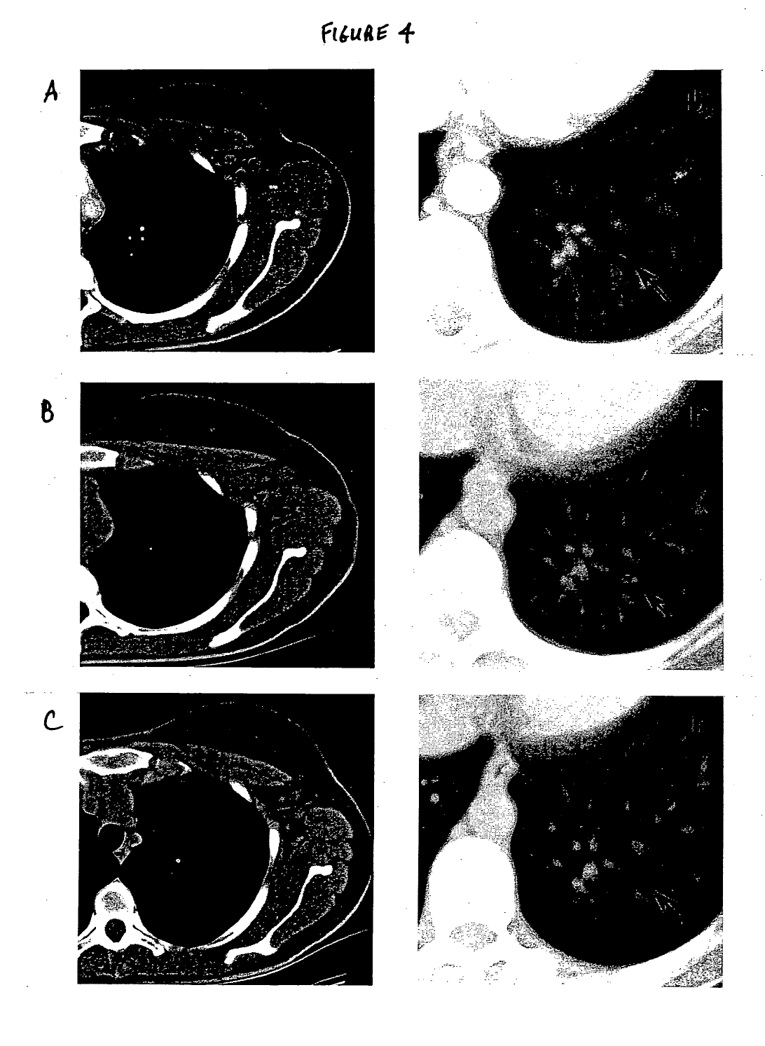

Association of increased gp100-reactive T cells after HDI and clinical responses in M335: A similar result was observed in M335, a 31 year old female who had initially presented with a 0.65 mm primary lesion on her right thigh. Six years later she developed right inguinal lymph node involvement, which was resected, and she received treatment for one year with the immunomodulatory agent, levamisole (Quirt, 1991, supra). Subsequently, she developed two subcutaneous metastases and was treated with HDI and 10 months of SC IFN-α2b at low doses. One year later she developed a right axillary mass which was dissected and treated with adjuvant radiation. Shortly thereafter, she had involvement of the skin and dermis of the right breast and chest wall, which was treated by mastectomy and local radiation. She was then enrolled in the melanoma vaccine trial, at which time she had developed multiple small melanotic skin metastases over the right chest but no detectable systemic disease otherwise (FIGS. 4a, d). Over the 12 weeks of the schedule of vaccine injections, she developed a 4 cm mass in the scar line of the mastectomy (not shown) and adenopathy in the left axilla (FIG. 4b) and cervical region. In addition, lung nodules were found, compatible with metastases (FIG. 4e). Six weeks after the last vaccine injection, she was started on HDI. Within 2 weeks, the palpable masses in the chest wall and left axilla had disappeared, as confirmed by the CT scan taken 2 months after completing HDI (FIG. 4c). Radiologic evidence of lung metastases (FIG. 4e) also disappeared (FIG. 4f). The patient has again been maintained on SC IFNα2a and, at the time of her last clinic visit, had no evidence of systemic metastases, except for the skin deposits. Interestingly, many of these had developed evidence of local vitiligo suggestive of auto-immune destruction of nearby normal melanocytes.

Similar to M166, M335 transiently responded to vaccination, as measured by tetramers and ELISPOT assays, but this response was lost by the time that HDI was instituted (FIG. 1b, FIG. 5). However, within 2 weeks of starting HDI, and concomitant with the observed clinical response (FIG. 4), the frequency of IFN-γ producing gp100-reactive T cells increased to ˜1/351 and the percentage of tetramer-staining CD8⁺ T cells increased to ˜7% of cultured PBMC by the third week of HDI. Elevated responses in these assays were maintained for at least one month after completing HDI (FIG. 1b and FIG. 5).

HDI alters the quality of the anti-tumor T cell response: The frequency of gp100-reactive T cells recalled by HDI was not significantly different from the frequency that was found in response to the tumor vaccine (Table 1, columns 7 and 8; FIG. 1; FIG. 3a,b; FIG. 5). It was hypothesized that the anti-tumor response recalled by HDI may be more potent than the response that developed after vaccination to account for the therapeutic effects seen in M166 and M335. It is generally believed that TH1/TC1 responses that result in the activation of cytotoxic T cells (CTLs) able to kill tumor cells are required for optimal anti-tumor immunity. Although IFN-γ production, as measured in the ELISPOT assays, is a surrogate marker for CD8⁺ CTL function, we directly examined the ability of gp100-reactive T cells from M166 and M335, during vaccination or during HDI, to kill targets expressing HLA-A*0201 molecules and gp100 peptides. Since melanoma cell lines from these patients were not available, peptide-loaded T2 cells were used as targets. T2 cells express complexes of peptides and HLA-A*0201 molecules on their cell surface only when HLA-A*0201 binding peptides are provided, because of a genetically defective TAP-transporter system. If gp100-reactive T cells are unable to kill gp100 peptide loaded T2 cells, it seems unlikely they could kill autologous melanoma cells with a much lower surface density of gp100 peptide-HLA-A*0201 complexes.

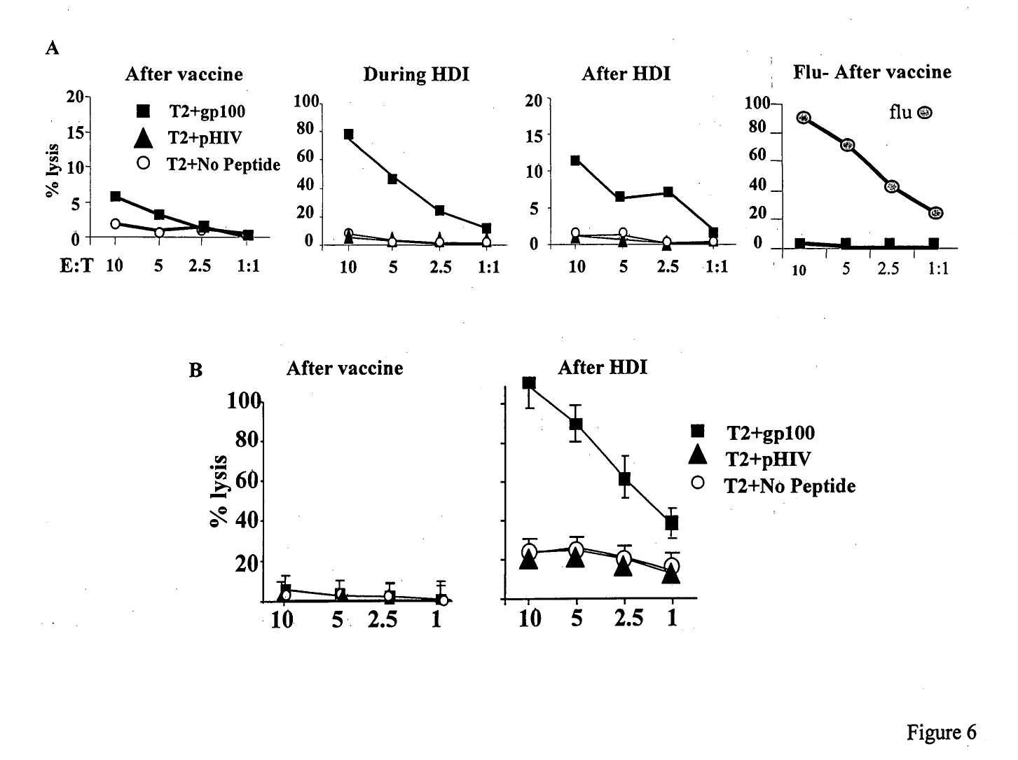

Despite the similar frequencies of tetramer staining and IFN-Y producing gp100-reactive T cells, there were striking differences in the ability to kill gp100-peptide loaded T2 cells after HDI. Tumor-reactive T cells activated by vaccination alone were unable to kill gp100 peptide-loaded T2 cells (FIG. 6a for M166 and FIG. 6b for M335, graphs “After vaccine”). However, gp100-reactive T cells during and after HDI from both patients were potent killers of gp100 peptide-loaded T2 cells (80% lysis at an E:T ratio of 10:1) (FIG. 6) This level of killing was comparable to that observed with FLU-stimulated T cells and FLU peptide-loaded T2 targets, performed at the same time (FIG. 6, graph “Flu-After vaccine).

In these examples, we have shown that HDI can increase both the frequency of tumor-reactive T cells initially activated by a cancer vaccine and the ability of these cells to kill tumor-antigen bearing targets.

It was observed that the number of tumor-reactive T cells measured by tetramers was often higher than found using the ELISPOT assays (compare FIGS. 1, 3, and 5). Such discrepancies have been noted before and may be due to T cells that are anergic or senescent or make TH2/TC2 cytokines, rather than IFN-γ, in response to peptide stimulation. It was also determined that a significant number of peptide-reactive T cells undergo activation-induced cell death upon re-stimulation by peptides in the ELISPOT plate and this phenomenon could also partially account for the lower numbers of antigen-specific cells found in the ELISPOT assays.

IFN-α is one of the oldest cytokines that has been characterized and used for immunotherapeutic purposes. It has pleiotropic effects on immune responses. However, it is unclear how HDI is acting to so strikingly affect the vaccine-induced immune responses. IFN-α increases the level of MHC expression on both melanoma cells and professional APCs such as dendritic cells (DCs). Consequently, residual melanoma cells in the patient may become able to directly activate g100-reactive T cells previously activated by the vaccine. Alternatively these T cells may be reactivated by DCs that indirectly present gp100 antigens that have been shed by residual melanoma cells. IFN-α is also known to prevent activation-induced cell death of T cells. If gp100-reactive T cells are being chronically activated by gp100 antigens in vivo, the numbers of these cells may be limited by ongoing apoptosis. Since the number of antigen-specific T cells represents the difference between the number that are proliferating and the number that are dying, apoptotic blockade would lead to increased numbers of tumor-reactive T cells. It has been shown that IFN-α causes bystander proliferation of CD8⁺ T cells, which may be another mechanism whereby gp100-reactive T cells reappear in the blood after HDI. This effect has recently been shown to be mediated indirectly through IL-15 possibly released by dendritic and stromal cells in response to IFN-α, which is consistent with our inability to mimic the results by directly adding IFN-α to T cell cultures.

The more potent responses seen in the in vitro CTL assays were mirrored in the clinical responses of the patients. M335 especially had suffered disease progression after IFN-α alone, and during vacination, but had a remarkable clinical response when HDI was administered after vaccination (FIG. 4). The mechanism by which the anti-tumor responses were made more potent by HDI is unclear. Although IFN-α is known to activate the lytic machinery and make T cells more potent CTLs, increased CTL activity in our experiments was noted 8 days after the cells had been removed from the patient and cultured in the absence of IFN-α. The effect is due to an in vivo process and non-cytotoxic gp100-reactive T cells have been induced by vaccines into potent CTLs by the addition of IFN-α to in vitro cultures.

While the present invention has been described in terms of the preferred embodiments, it is understood that variations and modifications will occur to those skilled in the art. Therefore, it is intended that the appended claims cover all such equivalent variations that come within the scope of the invention as claimed.

TABLE 1
Patient characteristics

| | | | | | | Peak frequency of | Peak frequency |
|---|---|---|---|---|---|---|---|
| | | Time from last | | | | gp 100-reactive T | of gp 100- |
| Patient | | vaccination to | Initial | Disease at | Current | cells during | reactive T cells |
| No. | Age/Sex | IFN-α | disease | time of IFN-α | Status | vaccination* | during HDI |
| M136 | 52/M | 8 months | Lung, LN** | NED*** | NED | 1/5 × 10⁴ | 1/1 × 10⁵ |
| M302 | 53/F | 3 months | Skin | NED | NED | 1/510 | 1/263 |
| | | | metastases | | | | |
| M246 | 47/F | 7 months | LN | NED | NED | 1/1 × 10⁵ | 1/1 × 10⁵ |
| M237 | 49/M | 8 months | LN | NED | NED | 1/6667 | 1/1667 |
| M166 | 33/M | 6 months | Mesenteric | Gluteal mass | Clinical | 1/6270 | 1/1111 |
| | | | Mass | | regression | | |
| M335 | 32/F | 1.5 months | LN, skin, | LNs, skin, | Clinical | 1/588 | 1/351 |
| | | | breast | lung | regression | | |
| M260 | 64/M | 17 months | LN | Lung | Lung (no | 1/2 × 10⁴ | 1/1 × 10⁵ |
| | | | | | change) | | |

*The peak frequency was the highest number of spots during at any time-point during active vaccination. ELISPOT assays wereperformed as described in the materials and methods and the average of three replicate wells is reported as 1/(average spot number/10⁵ plated cells).
**LN = lymph node
***NED = no evaluable disease

TABLE 2
Toxicity, treatment delays, and dose reductions
in patients receiving HDI after vaccination.

| | Grade 3* | Grade 2 | Total |
|---|---|---|---|
| Constitutional Symptoms | 1/7 | 3/7 | 4/7 |
| Vitiligo | 0/7 | 1/7 | 1/7 |
| Elevated Liver Function | 1/7 | 4/7 | 5/7 |
| Tests | | | |
| Granulocytopenia/leukopenia | 1/7 | 6/7 | 7/7 |
| Neurologic Toxicity | 1/7 | 1/7 | 2/7 |
| Dose reduction | | | 7/7 |
| Dose Delay | | | 7/7 |

*The delivery of HDI was modified for each patient on the basis of common toxicity criteria, ¹⁵with 4 being the most severe, necessitating stopping treatment. A 33% reduction of dosage occurred after the first treatment interruption and a 66% reduction from baseline dose occurred after the second. No patients had a third treatment interruption that would also have required removal from treatment.

What is claimed is:

1. A method for treating cancer comprising:
a) administering to a host a composition containing a tumor antigen, fragment thereof or nucleic acid encoding the tumor antigen such that the host develops an immune response against the tumor antigen; and,
b) subsequently administering to the host a high dose of a cytokine;
whereby the combination of steps a) and b) provides an enhanced T cell response in the host relative to that which occurs following step a) alone.

2. The method of claim 1 wherein the tumor antigen is administered as a polypeptide or peptide.

3. The method of claim 1 wherein the composition comprises a nucleic acid encoding a tumor antigen.

4. The method of claim 3 wherein the nucleic acid is contained within a plasmid or a viral vector.

5. The method of claim 4 wherein the viral vector is selected from the group consisting of poxvirus, adenovirus, retrovirus, herpesvirus, and adeno-associated virus.

6. The method of claim 5 wherein the viral vector is a poxvirus selected from the group consisting of vaccinia, NYVAC, MVA, avipox, canarypox, ALVAC, ALVAC(2), fowlpox, and TROVAC.

7. The method of claim 6 wherein the viral vector is a poxvirus selected from the group consisting of NYVAC, ALVAC, and ALVAC(2).

8. The method of claim 1 wherein the cytokine is IFN.

9. The method of claim 8 wherein the cytokine is IFN-α.

10. The method of claim 9 wherein the cytokine is IFN-α2b.

11. The method of claim 1 wherein the tumor antigen is selected from the group consisting of gp100, MART-1/Melan A, gp75/TRP-1, tyrosinase, NY-ESO-1, melanoma proteoglycan, a MAGE antigen, a BAGE antigen, a GAGE antigen, RAGE antigen, N-acetylglucosaminyltransferase-V, p15, β-catenin, MUM-1, cyclin dependent kinase-4, p21 -ras, BCR-abl, p53, p185 HER2/neu, epidermal growth factor receptor, carcinoembryonic antigen, modified carcinoembryonic antigen, carcinoma-associated mutated mucins, an Epstein Barr Virus EBNA gene product, papilloma virus E7, papilloma virus E6, prostate specific antigen, prostate specific membrane antigen, KSA, kinesin 2, HIP-55, TGFβ-1 anti-apoptotic factor, tumor protein D52, H1FT, an NY-BR antigen, fragments thereof, and derivatives thereof.

12. The method of claim 11 wherein the tumor antigen is selected from the group consisting of gp100, MAGE-1, MAGE-2, MAGE-3, MAGE-4, MAGE-6, MAGE-12, MAGE-51, GAGE-1, GAGE-2, RAGE-1, NY-BR-1, NY-BR-62, NY-BR-75, NY-BR-85, NY-BRP-87, and NY-BR-96.

13. The method of claim 12 wherein the tumor antigen is gp100.

14. The method of claim 1 wherein the composition comprises an poxviral vector encoding a tumor antigen or a fragment thereof and the cytokine is a T cell activating cytokine.

15. The method of claim 14 wherein poxviral vector is an ALVAC vector and the T cell activating cytokine is IFN.

16. The method of claim 15 wherein the T cell activating cytokine is IFNα.

17. The method of claim 16 wherein the T cell activating cytokine is IFNα2b.

18. The method of claim 17 wherein IFNα2b is administered at at least 10 MU/m²/d IV at least two times per week for at least two weeks.

19. The method of claim 18 wherein IFNα2b is administered at at least 10 MU/m²/d IV at least three times per week for at least two weeks.

20. The method of claim 19 wherein IFNα2b is administered at at least 10 MU/m²/d IV at least four times per week for at least two weeks.

21. The method of claim 20 wherein IFNα2b is administered at at least 10 MU/m²/d IV at least five times per week for at least two weeks.

22. The method of claim 21 wherein IFNα2b is administered at at least 20 MU/m²/d IV at least five times per week for at least four weeks.
